(19) United States
(12) Patent Application Publication
(10) Pub. No.: US 2024/0153632 A1
(43) Pub. Date: May 9, 2024

(54) COMPUTER-IMPLEMENTED METHODS AND SYSTEMS FOR QUANTITATIVELY DETERMINING A CLINICAL PARAMETER

(71) Applicant: Hoffmann-La Roche Inc., Little Falls, NJ (US)

(72) Inventor: Marco GANZETTI, Basel (CH)

(21) Appl. No.: 18/282,505

(22) PCT Filed: Mar. 30, 2022
(86) PCT No.: PCT/EP2022/058488

§ 371 Date: Sep. 15, 2023

(30) Foreign Application Priority Data
Mar. 30, 2021 (EP) 21166118.6

(51) Int. Cl. G16H 50/20; A61B 5/00; A61B 5/11; G06N 20/00

(52) U.S. Cl. CPC G16H 50/20; A61B 5/1127; A61B 5/4082; A61B 5/4566; A61B 5/4842; A61B 5/7435; G06N 20/00; A61B 2560/0437

(57) ABSTRACT

A computer-implemented method for quantitatively determining a clinical parameter indicative of a status or progression of a disease comprises the steps of: providing a distal motor test to a user of a mobile device, the mobile device having a touchscreen display, wherein providing the distal motor test to the user of the mobile device comprises: causing the touchscreen display of the mobile device to display an image comprising: a reference start point, a reference end point, and indication of a reference path to be traced between the start point and the end point; receiving an input from the touchscreen display of the mobile device, the input indicative of a test path traced by a user attempting to trace the reference path on the display of the mobile device, the test path comprising: a test start point, a test end point, and a test path traced between the test start point and the test end point; and extracting digital biomarker feature data from the received input, the digital biomarker feature data comprising: a deviation between the test end point and the reference end point; a deviation between the test start point and the reference start point; and/or a deviation between the test start point and the reference end point; and wherein: the extracted digital biomarker feature data is the clinical parameter; or the method further comprises calculating the clinical parameter from the extracted biomarker feature data.

### TECHNICAL FIELD

The present invention relates to the field of digital assessment of diseases. In particular, the present invention relates to computer-implemented methods and systems for quantitatively determining a clinical parameter indicative of the status or progression of a disease. The computer-implemented methods and systems may be used for determining an expanded disability status scale (EDSS) indicative of multiple sclerosis, a forced vital capacity indicative of spinal muscular atrophy, or a total motor score (TMS) indicative of Huntington's disease.

### BACKGROUND ART

Disease and, in particular, neurological diseases require intensive diagnostic measures for disease management. After the onset of the disease, theses disease, typically, are progressive diseases and need to be evaluated by staging system in order to determine the precise status. Prominent examples among those progressive neurological diseases, there are multiple sclerosis (MS), Huntington's Disease (HD) and spinal muscular atrophy (SMA).

Currently, the staging of such disease requires great efforts and is cumbersome for the patients which need to go to medical specialists in hospitals or doctor's offices. Moreover, staging requires experience at the end of the medical specialist and is often subjective and based on personal experience and judgement. Nevertheless, there are some parameters from disease staging which are particularly useful for the disease management. Moreover, there are other cases such as in SMA were a clinically relevant parameter such as the forced vital capacity needs to be determined by special equipment, i.e. spirometric devices.

For all of these cases, it might be helpful to determine surrogates. Suitable surrogates include biomarkers and, in particular, digitally acquired biomarkers such as performance parameters from tests which am at determining performance parameters of biological functions that can be correlated to the staging systems or that can be surrogate markers for the clinical parameters.

Correlations between the actual clinical parameter of interest, such as a score or other clinical parameter, can be derived from data by various methods.

### SUMMARY OF THE INVENTION

A first aspect of the present invention provides a computer-implemented method for quantitatively determining a clinical parameter indicative of a status or progression of a disease, the computer-implemented method comprising: providing a distal motor test to a user of a mobile device, the mobile device having a touchscreen display, wherein providing the distal motor test to the user of the mobile device comprises: causing the touchscreen display of the mobile device to display an image comprising: a reference start point, a reference end point, and indication of a reference path to be traced between the start point and the end point; receiving an input from the touchscreen display of the mobile device, the input indicative of a test path traced by a user attempting to trace the reference path on the display of the mobile device, the test path comprising: a test start point, a test end point, and a test path traced between the test start point and the test end point; and extracting digital biomarker feature data from the received input, the digital biomarker feature data comprising: a deviation between the test end point and the reference end point; a deviation between the test start point and the reference start point; and/or a deviation between the test start point and the reference end point; and wherein: the extracted digital biomarker feature data is the clinical parameter; or the method further comprises calculating the clinical parameter from the extracted biomarker feature data.

A second aspect of the present invention provides system for quantitatively determining a clinical parameter indicative of a status or progression of a disease, the system including: a mobile device having a touchscreen display, a user input interface, and a first processing unit; and a second processing unit; wherein: the mobile device is configured to provide a distal motor test to a user thereof, wherein providing the distal motor test comprises: the first processing unit causing the touchscreen display of the mobile device to display an image comprising: a reference start point, a reference end point, and indication of a reference path to be traced between the start point and the end point; the user input interface is configured to receive from the touchscreen display, an input indicative of a test path traced by a user attempting to trace the reference path on the display of the mobile device, the test path comprising: a test start point, a test end point, and a test path traced between the test start point and the test end point; and the first processing unit or the second processing unit is configured to extract digital biomarker feature data from the received input, the digital biomarker feature data comprising: a deviation between the test end point and the reference end point; and/or a deviation between the test start point and the test end point; and wherein: the extracted digital biomarker feature data is the clinical parameter; or the first processing unit or the second processing unit is further configured to calculate the clinical parameter from the extract digital biomarker feature data.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarized here and without excluding further possible embodiments, the following embodiments may be envisaged.

Embodiment 1: A computer-implemented method for quantitatively determining a clinical parameter which is indicative of the status or progression of a disease, the computer-implemented method comprising:
providing a distal motor test to a user of a mobile device, the mobile device having a touchscreen display, wherein providing the distal motor test to the user of the mobile device comprises:
   causing the touchscreen display of the mobile device to display a test image;
receiving an input from the touchscreen display of the mobile device, the input indicative of an attempt by a user to place a first finger on a first point in the test image and a second finger on a second point in the test image, and to pinch the first finger and the second finger together, thereby bringing the first point and the second point together;
extracting digital biomarker feature data from the received input.

Embodiment 2: A computer-implemented method according to embodiment 1, wherein:
the first point and the second point are specified and/or identified in the test image.

Embodiment 3: A computer-implemented method according to embodiment 1, wherein:
the first point is not specified in the test image, and is defined as the point where the first finger touches the touchscreen display; and
the second point is not specified in the test image, and is defined as the point where the second finger touches the touchscreen display.

Embodiment 4: A computer-implemented method according to any one of embodiments 1 to 3, wherein:
the extracted digital biomarker feature data is the clinical parameter.

Embodiment 5: A computer-implemented method according to any one of embodiments 1 to 3, further comprising:
calculating the clinical parameter from the extracted digital biomarker feature data.

Embodiment 6: The computer-implemented method of any one of embodiments 1 to 5, wherein:
the received input includes:
   data indicative of the time when the first finger leaves the touchscreen display;
   data indicative of the time when the second finger leaves the touchscreen display.

Embodiment 7: The computer-implemented method of embodiment 6, wherein:
the digital biomarker feature data includes the difference between the time when the first finger leaves the touchscreen display and the time when the second finger leaves the touchscreen display.

Embodiment 8: The computer-implemented method of any one of embodiments 1 to 7, wherein:
the received input includes:
   data indicative of the time when the first finger initially touches the first point;
   data indicative of the time when the second finger initially touches the second point.

Embodiment 9: The computer-implemented method of embodiment 8, wherein:
the digital biomarker feature data includes the difference between the time when the first finger initially touches the first point and the time when the second finger initially touches the second point.

Embodiment 10: The computer-implemented method of embodiment 8 or embodiment 9, wherein:
the digital biomarker feature data includes the difference between:
   the earlier of the time when the first finger initially touches the first point, and the time when the second finger initially touches the second point; and
   the later of the time when the first finger leaves the touchscreen display and the time when the second finger leaves the touchscreen display.

Embodiment 11: The computer-implemented method of any one of embodiments 1 to 10, wherein:
the received input includes:
   data indicative of the location of the first finger when it leaves the touchscreen display; and
   data indicative of the location of the second finger when it leaves the touchscreen display.

Embodiment 12: The computer-implemented method of embodiment 11, wherein:
the digital biomarker feature data includes the distance between the location of the first finger when it leaves the touchscreen display and the location of the second finger when it leaves the touchscreen display.

Embodiment 13: The computer-implemented method of any one of embodiments 1 to 12, wherein:
the received input includes:
   data indicative of the first path traced by the first finger from the time when it initially touches the first point to the time when it leaves the touchscreen, the data including a first start point, a first end point, and a first path length; and
   data indicative of the second path traced by the second finger from the time when it initially touches the second point to the time when it leaves the touchscreen, the data including a second start point, a second end point, and a second path length.

Embodiment 14: The computer-implemented method of embodiment 13, wherein:
the digital biomarker feature data includes a first smoothness parameter, the first smoothness parameter being the ratio of the first path length and the distance between the first start point and the first end point;
the digital biomarker feature data includes a second smoothness parameter, the second smoothness parameter being the ratio of the second path length and the distance between the second start point and the second end point.

Embodiment 15: The computer-implemented method of any one of embodiments 1 to 14, wherein:
the method comprises:
   receiving a plurality of inputs from the touchscreen display of the mobile device, each of the plurality of inputs indicative of a respective attempt by a user to place a first finger on a first point in the test image and a second finger on a second point in the test image, and to pinch the first finger and the second finger together, thereby bringing the first point and the second point together; and
   extracting a respective piece of digital biomarker feature data from each of the plurality of received inputs, thereby generating a respective plurality of pieces of digital biomarker feature data.

Embodiment 16: The computer implemented method of embodiment 15, wherein:
the method further comprises:
   determining a subset of the respective pieces of digital biomarker feature data which correspond to successful attempts.

The purpose of the present invention is to use a simple mobile device-based test to determine progress of a disease which affects a user's motor control. In view of that, the success of a test preferably depends on the extent to which a user is successfully able to bring the first point and the second point together without lifting their fingers from the touchscreen display surface. The step of determining whether an attempt has been successful preferably includes determining a distance between the location where the first finger leaves the touchscreen display and the location where the second finger leaves the touchscreen display. A successful attempt may be defined as an attempt in which this distance falls below a predetermined threshold. Alternatively, the step of determining whether an attempt has been successful may include determining a distance from a midpoint between the initial location of the first point and an initial location of the second point, of the location where the first finger leaves the touchscreen display, and a distance from a midpoint between the initial location of the first point and an initial location of the second point, of the location where the second finger leaves the touchscreen display. A successful attempt may be defined as an attempt where the average of the two distances is below a predetermined threshold or alternatively, an attempt where both of the distances are below a predetermined threshold.

Embodiment 17: The computer-implemented method of any one of embodiments 1 to 14, wherein:
the method comprises:
   receiving a plurality of inputs from the touchscreen display of the mobile device, each of the plurality of inputs indicative of a respective attempt by a user to place a first finger on a first point in the test image and a second finger on a second point in the test image, and to pinch the first finger and the second finger together, thereby bringing the first point and the second point together;
   determining a subset of the plurality of received inputs which correspond to successful attempts; and
   extracting a respective piece of digital biomarker feature data from each of the determined subset of plurality of received inputs, thereby generating a respective plurality of pieces of digital biomarker feature data.

Embodiment 18: The computer-implemented method of any one of embodiments 15 to 17, wherein:
the method further comprises deriving a statistical parameter from either:
   the plurality of pieces of digital biomarker feature data, or
   the determined subset of the respective pieces of digital biomarker feature data which correspond to successful attempts.

Embodiment 19: The computer-implemented method of embodiment 18, wherein:
the statistical parameter includes:
   the mean of the plurality of pieces of digital biomarker feature data; and/or
   the standard deviation of the plurality of pieces of digital biomarker feature data; and/or
   the kurtosis of the plurality of pieces of digital biomarker feature data;
   the median of the plurality of pieces of digital biomarker feature data;
   a percentile of the plurality of pieces of digital biomarker feature data.

The percentile may be the 5%, 10%, 15%, 20%, 25%, 30%, 33%, 35%, 40%, 45%, 50%, 55%, 60%, 65%, 66%, 67%, 70%, 75%, 80%, 85%, 90%, 95%.

Embodiment 20: The computer-implemented method of any one of embodiments 14 to 19, wherein:
the plurality of received inputs are received in a total time consisting of a first time period followed by a second time period;
the plurality of received inputs includes:
   a first subset of received inputs received during the first time period, the first subset of received inputs having a respective first subset of extracted pieces of digital biomarker feature data; and
   a second subset of inputs received during the second time period, the second subset of received inputs having a respective second subset of extracted pieces of digital biomarker feature data;
the method further comprises:
   deriving a first statistical parameter corresponding to the first subset of extracted pieces of digital biomarker feature data;
   deriving a second statistical parameter corresponding to the second subset of extracted pieces of digital biomarker feature data; and
   calculating a fatigue parameter by calculating the difference between the first statistical parameter and the second statistical parameter, and optionally dividing the difference by the first statistical parameter.

Embodiment 21: The computer-implemented method of embodiment 20, wherein:
the first time period and the second time period are the same duration.

Embodiment 22: The computer-implemented method of any one of embodiments 15 to 21, wherein:
the plurality of received inputs includes:
   a first subset of received inputs, each indicative of an attempt by a user to place a first finger of their dominant hand on a first point in the test image and a second finger of their dominant hand on a second point in the test image, and to pinch the first finger of their dominant hand and the second finger of their dominant hand together, thereby bringing the first point and the second point together, the first subset of received inputs having a respective first subset of extracted pieces of digital biomarker feature data; and
   a second subset of received inputs, each indicative of an attempt by a user to place a first finger of their non-dominant hand on a first point in the test image and a second finger of their non-dominant hand on a second point in the test image, and to pinch the first finger of their non-dominant hand and the second finger of their non-dominant hand together, thereby bringing the first point and the second point together, the second subset of received inputs having a respective second subset of extracted pieces of digital biomarker feature data;
the method further comprises:
   deriving a first statistical parameter corresponding to the first subset of extracted pieces of digital biomarker feature data;
   deriving a second statistical parameter corresponding to the second subset of extracted pieces of digital biomarker feature data; and
   calculating a handedness parameter by calculating the difference between the first statistical parameter and the second statistical parameter, and optionally dividing the difference by the first statistical parameter or the second statistical parameter.

Embodiment 23: The computer-implemented method of any one of embodiments 15 to 22, wherein:
the method further comprises:
   determining a first subset of the plurality of received inputs corresponding to user attempts in which only the first finger and the second finger contact the touchscreen display;
   determining a second subset of the plurality of received inputs corresponding to user attempts in which either only one finger, or three or more fingers contact the touchscreen display; and
the digital biomarker feature data comprises:
   the number of received inputs in the first subset of received inputs; and/or
   the proportion of the total number of received inputs which are in the first subset of received inputs.

Embodiment 24: The computer-implemented method of any one of embodiments 15 to 23, wherein:
each received input of the plurality of received inputs includes:
   data indicative of the time when the first finger initially touches the first point;
   data indicative of the time when the second finger initially touches the second point
   data indicative of the time when the first finger leaves the touchscreen display; and
   data indicative of the time when the second finger leaves the touchscreen display;
the method further includes, for each successive pair of inputs, determining the time interval between:
   the later of the time at which the first finger leaves the touchscreen display and the time at which the second finger leaves the touchscreen display, for the first of the successive pair of received inputs; and
   the earlier of the time at which the first finger initially touches the first point and the time at which the second finger touches the second point, for the second of the successive pair of received inputs.
the extracted digital biomarker feature data comprises:
   the set of the determined time intervals;
   the mean of the determined time intervals;
   the standard deviations of the determined time intervals; and/or
   the kurtosis of the determined time intervals.

Embodiment 25: The computer-implemented method of any one of embodiments 1 to 24, wherein:
the method further comprises obtaining acceleration data.

Embodiment 26: The computer-implemented method of embodiment 25, wherein:
the acceleration data includes one or more of the following:
   (a) a statistical parameter derived from the magnitude of the acceleration throughout the duration of the whole test;
   (b) a statistical parameter derived from the magnitude of the acceleration only during periods where the first finger, the second finger, or both fingers are in contact with the touchscreen display; and
   (c) a statistical parameter of the magnitude of the acceleration only during periods where no finger is in contact with the touchscreen display.

Embodiment 27: The computer-implemented method of embodiment 20, wherein:
the statistical parameter includes one or more of the following:
   the mean;
   the standard deviation;
   the median;
   the kurtosis; and
   a percentile.

Embodiment 28: The computer-implemented method of any one of embodiments 25 to 27, wherein:
the acceleration data includes a z-axis deviation parameter, wherein determining the z-axis deviation parameter comprises:
   for each of a plurality of points in time, determining the magnitude of the z-component of the acceleration, and calculating the standard deviation of the z-component of the acceleration over all of the points in time, wherein the z-direction is defined as the direction which is perpendicular to a plane of the touchscreen display.

Embodiment 29: The computer-implemented method of any one of embodiments 25 to 28, wherein:
the acceleration data includes a standard deviation norm parameter, wherein determining the standard deviation norm parameter comprises:
   for each of a plurality of points in time, determining the magnitude of the x-component of the acceleration, and calculating the standard deviation of the x-component of the acceleration over all of the points in time;
   for each of a plurality of points in time, determining the magnitude of the y-component of the acceleration, and calculating the standard deviation of the y-component of the acceleration over all of the points in time;
   for each of a plurality of points in time, determining the magnitude of the z-component of the acceleration, and calculating the standard deviation of the z-component of the acceleration over all of the points in time, wherein the z-direction is defined as the direction which is perpendicular to a plane of the touchscreen display; and
   calculating the norm of the respective standard deviations of the x-component, the y-component, and the z-component by adding them in quadrature.

Embodiment 30: The computer-implemented method of any one of embodiments 25 to 29, wherein:
the acceleration data includes a horizontality parameter, wherein determining the horizontality parameter includes:
   for each of a plurality of points in time, determining:
      a magnitude of the acceleration; and
      a magnitude of the z-component of the acceleration, wherein the z-direction is defined as the direction which is perpendicular to a plane of the touchscreen display;
      the ratio of the z-component of the acceleration and the magnitude of the acceleration;
   determining the mean of the determined ratio over the plurality of points in time.

Embodiment 31: The computer-implemented method of any one of embodiments 25 to 30, wherein:
the acceleration data includes an orientation stability parameter, wherein determining the orientation stability parameter includes:
   for each of a plurality of points in time, determining:
      a magnitude of the acceleration; and
      a magnitude of the z-component of the acceleration, wherein the z-direction is defined as the direction which is perpendicular to a plane of the touchscreen display;
      the ratio of the z-component of the acceleration and the magnitude of the acceleration value;
   determining the standard deviation of the determined ratio over the plurality of points in time.

Embodiment 32: The computer-implemented method of any one of embodiments 1 to 31, further comprising:
applying at least one analysis model to the digital biomarker feature data or a statistical parameter derived from the digital biomarker feature data; and
predicting a value of the at least one clinical parameter based on the output of the at least one analysis model.

Embodiment 33: The computer-implemented method of embodiment 32, wherein:
the analysis model comprises a trained machine learning model.

Embodiment 34: The computer-implemented method of embodiment 33, wherein:
the analysis model is a regression model, and the trained machine learning model comprises one or more of the following algorithms:
   a deep learning algorithm;
   k nearest neighbours (kNN);
   linear regression;
   partial last-squares (PLS);
   random forest (RF); and
   extremely randomized trees (XT).

Embodiment 35: The computer implemented method of 33, wherein:
the analysis model is a classification model, and the trained machine learning model comprises one or more of the following algorithms:
   a deep learning algorithm;
   k nearest neighbours (kNN);
   support vector machines (SVM);
   linear discriminant analysis;
   quadratic discriminant analysis (QDA);
   naïve Bayes (NB);
   random forest (RF); and
   extremely randomized trees (XT).

Embodiment 36: The computer-implemented method of any one of embodiments 1 to 35, wherein:
the disease whose status is to be predicted is multiple sclerosis and the clinical parameter comprises an expanded disability status scale (EDSS) value,
the disease whose status is to be predicted is spinal muscular atrophy and the clinical parameter comprises a forced vital capacity (FVC) value, or
wherein the disease whose status is to be predicted is Huntington's disease and the clinical parameter comprises a total motor score (TMS) value.

Embodiment 37: The computer-implemented method of any one of embodiments 1 to 36, wherein:
the method further comprises determining the at least one analysis model, wherein determining the at least one analysis model comprises:
   (a) receiving input data via at least one communication interface, wherein the input data comprises a set of historical digital biomarker feature data, wherein the set historical digital biomarker feature data comprises a plurality of measured values indicative of the disease status to be predicted;
   (b) determining at least one training data set and at least one test data set from the input data set;
   (c) determining the analysis model by training a machine learning model comprising at least one algorithm with the training data set;
   (d) predicting the clinical parameter on the test data set using the determined analysis model;
   (e) determining performance of the determined analysis model based on the predicted target variable and a true value of the clinical parameter of the test data set.

Embodiment 38: The computer-implemented method of embodiment 37, wherein:
in step (c) a plurality of analysis models is determined by training a plurality of machine learning models with the training data set, wherein the machine learning models are distinguished by their algorithm, wherein in step d) a plurality of clinical parameters is predicted on the test data set using the determined analysis models, and
wherein in step (e) the performance of each of the determined analysis models is determined based on the predicted target variables and the true value of the clinical parameters of the test data set, wherein the method further comprises determining the analysis model having the best performance.

Embodiment 39: A system for quantitatively determining a clinical parameter which is indicative of a the status or progression of a disease, the system including:
a mobile device having a touchscreen display, a user input interface, and a first processing unit; and
a second processing unit;
wherein:
   the mobile device is configured to provide a distal motor test to a user thereof, wherein providing the distal motor test comprises:
      the first processing unit causing the touchscreen display of the mobile device to display a test image;
   the user input interface is configured to receive from the touchscreen display, an input indicative of an attempt by a user to place a first finger on a first point in the test image and a second finger on a second point in the test image, and to pinch the first finger and the second finger together, thereby bringing the first point and the second point together;
   the first processing unit or the second processing unit is configured to extract digital biomarker feature data from the received input.

Embodiment 40: The system of embodiment 39, wherein:
the first point and the second point are specified and/or identified in the test image.

Embodiment 41: The system of embodiment 39, wherein:
the first point is not specified in the test image, and is defined as the point where the first finger touches the touchscreen display; and
the second point is not specified in the test image, and is defined as the point where the second finger touches the touchscreen display.

Embodiment 42: The system of any one of embodiments 39 to 41, wherein:
the extracted digital biomarker feature data is the clinical parameter.

Embodiment 43: The system of any one of embodiments 39 to 41, wherein:
the first processing unit or the second processing unit is configured to calculate the clinical parameter from the extracted digital biomarker feature data.

Embodiment 44: The system of any one of embodiments 39 to 43, wherein:
the received input includes:
   data indicative of the time when the first finger leaves the touchscreen display;
   data indicative of the time when the second finger leaves the touchscreen display.

Embodiment 45: The system of embodiment 44, wherein:
the digital biomarker feature data includes the difference between the time when the first finger leaves the touchscreen display and the time when the second finger leaves the touchscreen display.

Embodiment 46: The system of any one of embodiments 39 to 45, wherein:
the received input includes:
   data indicative of the time when the first finger initially touches the first point;
   data indicative of the time when the second finger initially touches the second point.

Embodiment 47: The system of embodiment 46, wherein:
the digital biomarker feature data includes the difference between the time when the first finger initially touches the first point and the time when the second finger initially touches the second point.

Embodiment 48: The system of embodiment 46 or embodiment 47, wherein:
the digital biomarker feature data includes the difference between:
   the earlier of the time when the first finger initially touches the first point, and the time when the second finger initially touches the second point; and
   the later of the time when the first finger leaves the touchscreen display and the time when the second finger leaves the touchscreen display.

Embodiment 49: The system of any one of embodiments 39 to 48, wherein:
the received input includes:
   data indicative of the location of the first finger when it leaves the touchscreen display; and
   data indicative of the location of the second finger when it leaves the touchscreen display.

Embodiment 50: The system of embodiment 49, wherein:
the digital biomarker feature data includes the distance between the location of the first finger when it leaves the touchscreen display and the location of the second finger when it leaves the touchscreen display.

Embodiment 51: The system of any one of embodiments 39 to 50, wherein:
the received input includes:
   data indicative of the first path traced by the first finger from the time when it initially touches the first point to the time when it leaves the touchscreen, the data including a first start point, a first end point, and a first path length; and
   data indicative of the second path traced by the second finger from the time when it initially touches the second point to the time when it leaves the touchscreen, the data including a second start point, a second end point, and a second path length.

Embodiment 52: The system of embodiment 51, wherein:
the digital biomarker feature data includes a first smoothness parameter, the first smoothness parameter being the ratio of the first path length and the distance between the first start point and the first end point;
the digital biomarker feature data includes a second smoothness parameter, the second smoothness parameter being the ratio of the second path length and the distance between the second start point and the second end point.

Embodiment 53: The system of any one of embodiments 39 to 52, wherein:
the user input interface is configured to receive a plurality of inputs from the touchscreen display of the mobile device, each of the plurality of inputs indicative of a respective attempt by a user to place a first finger on a first point in the test image and a second finger on a second point in the test image, and to pinch the first finger and the second finger together, thereby bringing the first point and the second point together; and
the first processing unit or the second processing unit is configured to extract a respective piece of digital biomarker feature data from each of the plurality of received inputs, thereby generating a respective plurality of pieces of digital biomarker feature data.

Embodiment 54: The system of embodiment 53, wherein:
the first processing unit or the second processing unit is configured to determine a subset of the respective pieces of digital biomarker feature data which correspond to successful attempts.

Embodiment 55: The system of any one of embodiments 39 to 52, wherein:
the user input interface is configured to receive a plurality of inputs from the touchscreen display of the mobile device, each of the plurality of inputs indicative of a respective attempt by a user to place a first finger on a first point in the test image and a second finger on a second point in the test image, and to pinch the first finger and the second finger together, thereby bringing the first point and the second point together; and
the first processing unit or the second processing unit is configured to:
   determine a subset of the plurality of received inputs which correspond to successful attempts; and
   extract a respective piece of digital biomarker feature data from each of the determined subset of plurality of received inputs, thereby generating a respective plurality of pieces of digital biomarker feature data.

Embodiment 56: The system of any one of embodiments 53 to 55, wherein:
the first processing unit or the second processing unit is configured to derive a statistical parameter from either:
   the plurality of pieces of digital biomarker feature data, or
   the determined subset of the respective pieces of digital biomarker feature data which correspond to successful attempts.

Embodiment 57: The system of embodiment 56, wherein:
the statistical parameter includes:
   the mean of the plurality of pieces of digital biomarker feature data; and/or
   the standard deviation of the plurality of pieces of digital biomarker feature data; and/or
   the kurtosis of the plurality of pieces of digital biomarker feature data.

Embodiment 58: The system of any one of embodiments 53 to 57, wherein:
the plurality of received inputs are received in a total time consisting of a first time period followed by a second time period;
the plurality of received inputs includes:
   a first subset of received inputs received during the first time period, the first subset of received inputs having a respective first subset of extracted pieces of digital biomarker feature data; and
   a second subset of inputs received during the second time period, the second subset of received inputs having a respective second subset of extracted pieces of digital biomarker feature data; and
the first processing unit or the second processing unit is configured to:
   derive a first statistical parameter corresponding to the first subset of extracted pieces of digital biomarker feature data;
   derive a second statistical parameter corresponding to the second subset of extracted pieces of digital biomarker feature data; and
   calculate a fatigue parameter by calculating the difference between the first statistical parameter and the second statistical parameter, and optionally divide the difference by the first statistical parameter.

Embodiment 59: The system of embodiment 58, wherein:
the first time period and the second time period are the same duration.

Embodiment 60: The system of any one of embodiments 53 to 59, wherein:
the plurality of received inputs includes:
   a first subset of received inputs, each indicative of an attempt by a user to place a first finger of their dominant hand on a first point in the test image and a second finger of their dominant hand on a second point in the test image, and to pinch the first finger of their dominant hand and the second finger of their dominant hand together, thereby bringing the first point and the second point together, the first subset of received inputs having a respective first subset of extracted pieces of digital biomarker feature data; and
   a second subset of received inputs, each indicative of an attempt by a user to place a first finger of their non-dominant hand on a first point in the test image and a second finger of their non-dominant hand on a second point in the test image, and to pinch the first finger of their non-dominant hand and the second finger of their non-dominant hand together, thereby bringing the first point and the second point together, the second subset of received inputs having a respective second subset of extracted pieces of digital biomarker feature data; and
the first processing unit or the second processing unit is configured to:
   derive a first statistical parameter corresponding to the first subset of extracted pieces of digital biomarker feature data;
   derive a second statistical parameter corresponding to the second subset of extracted pieces of digital biomarker feature data; and
   calculate a handedness parameter by calculating the difference between the first statistical parameter and the second statistical parameter, and optionally divide the difference by the first statistical parameter or the second statistical parameter.

Embodiment 61: The system of any one of embodiments 53 to 60, wherein:
the first processing unit or the second processing unit is configured to:
   determine a first subset of the plurality of received inputs corresponding to user attempts in which only the first finger and the second finger contact the touchscreen display;
   determine a second subset of the plurality of received inputs corresponding to user attempts in which either only one finger, or three or more fingers contact the touchscreen display; and
the digital biomarker feature data comprises:
   the number of received inputs in the first subset of received inputs; and/or
   the proportion of the total number of received inputs which are in the first subset of received inputs.

Embodiment 62: The system of any one of embodiments 53 to 61, wherein:
each received input of the plurality of received inputs includes:
   data indicative of the time when the first finger initially touches the first point;
   data indicative of the time when the second finger initially touches the second point
   data indicative of the time when the first finger leaves the touchscreen display; and
   data indicative of the time when the second finger leaves the touchscreen display;
the first processing unit or the second processing unit is configured, for each successive pair of inputs, to determine the time interval between:
   the later of the time at which the first finger leaves the touchscreen display and the time at which the second finger leaves the touchscreen display, for the first of the successive pair of received inputs; and
   the earlier of the time at which the first finger initially touches the first point and the time at which the second finger touches the second point, for the second of the successive pair of received inputs.
the extracted digital biomarker feature data comprises:
   the set of the determined time intervals;
   the mean of the determined time intervals;
   the standard deviations of the determined time intervals; and/or
   the kurtosis of the determined time intervals.

Embodiment 63: The system of any one of embodiments 39 to 62, wherein:
the system further comprises an accelerometer configured to measure acceleration of the mobile device; and
either the first processing unit, the second processing unit, or the accelerometer is configured to generate acceleration data based on the measured acceleration.

Embodiment 64: The system of embodiment 63, wherein:
the acceleration data includes one or more of the following:
   (a) a statistical parameter derived from the magnitude of the acceleration throughout the duration of the whole test;
   (b) a statistical parameter derived from the magnitude of the acceleration only during periods where the first finger, the second finger, or both fingers are in contact with the touchscreen display; and
   (c) a statistical parameter of the magnitude of the acceleration only during periods where no finger is in contact with the touchscreen display.

Embodiment 65: The system of embodiment 64, wherein:
the statistical parameter includes one or more of the following:
   the mean;
   the standard deviation;
   the median;
   the kurtosis; and
   a percentile.

Embodiment 66: The system of any one of embodiments 63 to 65, wherein:
the acceleration data includes a z-axis deviation parameter, wherein determining the z-axis deviation parameter; and
the first processing unit or the second processing unit is configured to generate the z-axis deviation parameter by, for each of a plurality of points in time, determining the magnitude of the z-component of the acceleration, and calculating the standard deviation of the z-component of the acceleration over all of the points in time, wherein the z-direction is defined as the direction which is perpendicular to a plane of the touchscreen display.

Embodiment 67: The system of any one of embodiments 63 to 66, wherein:
the acceleration data includes a standard deviation norm parameter, wherein the first processing unit or the second processing unit is configured to determine the standard deviation norm parameter by:
   for each of a plurality of points in time, determining the magnitude of the x-component of the acceleration, and calculating the standard deviation of the x-component of the acceleration over all of the points in time;
   for each of a plurality of points in time, determining the magnitude of the y-component of the acceleration, and calculating the standard deviation of the y-component of the acceleration over all of the points in time;
   for each of a plurality of points in time, determining the magnitude of the z-component of the acceleration, and calculating the standard deviation of the z-component of the acceleration over all of the points in time, wherein the z-direction is defined as the direction which is perpendicular to a plane of the touchscreen display; and
   calculating the norm of the respective standard deviations of the x-component, the y-component, and the z-component by adding them in quadrature.

Embodiment 68: The system of any one of embodiments 63 to 67, wherein:
the acceleration data includes a horizontality parameter, wherein the first processing unit or the second processing unit is configured to determine the horizontality parameter by:
   for each of a plurality of points in time, determining:
      a magnitude of the acceleration; and
      a magnitude of the z-component of the acceleration, wherein the z-direction is defined as the direction which is perpendicular to a plane of the touchscreen display;
      the ratio of the z-component of the acceleration and the magnitude of the acceleration; and
   determining the mean of the determined ratio over the plurality of points in time.

Embodiment 69: The system of any one of embodiments 63 to 68, wherein:
the acceleration data includes an orientation stability parameter, wherein the first processing unit or the second processing unit is configured to determine the orientation stability parameter by:
   for each of a plurality of points in time, determining:
      a magnitude of the acceleration; and
      a magnitude of the z-component of the acceleration, wherein the z-direction is defined as the direction which is perpendicular to a plane of the touchscreen display;
      the ratio of the z-component of the acceleration and the magnitude of the acceleration value; and
   determining the standard deviation of the determined ratio over the plurality of points in time.

Embodiment 70: The system of any one of embodiments 39 to 69, wherein:
the second processing unit is configured to apply at least one analysis model to the digital biomarker feature data or a statistical parameter derived from the digital biomarker feature data, and to predict a value of the at least one clinical parameter based on an output of the at least one analysis model.

Embodiment 71: The system of embodiment 70, wherein:
the analysis model comprises a trained machine learning model.

Embodiment 72: The system of 71, wherein:
the analysis model is a regression model, and the trained machine learning model comprises one or more of the following algorithms:
   a deep learning algorithm;
   k nearest neighbours (kNN);
   linear regression;
   partial last-squares (PLS);
   random forest (RF); and
   extremely randomized trees (XT).

Embodiment 73: The system of embodiment 71, wherein:
the analysis model is a classification model, and the trained machine learning model comprises one or more of the following algorithms:
   a deep learning algorithm;
   k nearest neighbours (kNN);
   support vector machines (SVM);
   linear discriminant analysis;
   quadratic discriminant analysis (QDA);
   naïve Bayes (NB);
   random forest (RF); and
   extremely randomized trees (XT).

Embodiment 74: The system of any one of embodiments 39 to 73, wherein:
the disease whose status is to be predicted is multiple sclerosis and the clinical parameter comprises an expanded disability status scale (EDSS) value,
the disease whose status is to be predicted is spinal muscular atrophy and the clinical parameter comprises a forced vital capacity (FVC) value, or
wherein the disease whose status is to be predicted is Huntington's disease and the clinical parameter comprises a total motor score (TMS) value.

Embodiment 75: The system of any one of embodiments 39 to 74, wherein:
the first processing unit and the second processing unit are the same processing unit.

Embodiment 76: The system of any one of embodiments 39 to 74, wherein:
the first processing unit is separate from the second processing unit.

Embodiment 77: The system of any one of embodiments 39 to 76, further comprising a machine learning system for determining the at least one analysis model for predicting the clinical parameter indicative of a disease status, the machine learning system comprising:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the clinical parameter of the test data set using the determined analysis model, wherein the processing unit is configured for determining performance of the determined analysis model based on the predicted clinical parameter and a true value of the clinical parameter of the test data set, wherein the processing unit is the first processing unit or the second processing unit.

Embodiment 78: A computer-implemented method for quantitatively determining a clinical parameter which is indicative of a status or progression of a disease, the computer-implemented method comprising:
receiving an input from the mobile device, the input comprising:
   acceleration data from an accelerometer, the acceleration data comprising a plurality of points, each point corresponding to the acceleration at a respective time;
extracting digital biomarker feature data from the received input, wherein extracting the digital biomarker feature data includes:
   determining, for each of the plurality of points, a ratio of the total magnitude of the acceleration and the magnitude of the z-component of the acceleration at the respective time; and
   deriving a statistical parameter from the plurality of determined ratios, the statistical parameter including a mean, a standard deviation, a percentile, a median, and a kurtosis.

Embodiment 79: A system for quantitatively determining a clinical parameter which is indicative of a status or progression of a disease, the system including:
a mobile device having a an accelerometer, and a first processing unit; and
a second processing unit;
wherein:
   the accelerometer is configured to measure acceleration, and either the accelerometer, the first processing unit or the second processing unit is configured to generate acceleration data comprising a plurality of points, each point corresponding to the acceleration at a respective time;
   the first processing unit or the second processing unit is configured to extract digital biomarker feature data from the received input by:
      determining, for each of the plurality of points, a ratio of the total magnitude of the acceleration and the magnitude of the z-component of the acceleration at the respective time; and
      deriving a statistical parameter from the plurality of determined ratios, the statistical parameter including a mean, a standard deviation, a percentile, a median, and a kurtosis.

Embodiment 80: A computer-implemented method for quantitatively determining a clinical parameter indicative of a status or progression of a disease, the computer-implemented method comprising:
providing a distal motor test to a user of a mobile device, the mobile device having a touchscreen display, wherein providing the distal motor test to the user of the mobile device comprises:
   causing the touchscreen display of the mobile device to display an image comprising: a reference start point, a reference end point, and indication of a reference path to be traced between the start point and the end point;
receiving an input from the touchscreen display of the mobile device, the input indicative of a test path traced by a user attempting to trace the target path on the display of the mobile device, the test path comprising: a test start point, a test end point, and a test path traced between the test start point and the test end point;
extracting digital biomarker feature data from the received input, the digital biomarker feature data comprising:
   a deviation between the test end point and the reference end point;
   a deviation between the test start point and the reference start point; and/or
   a deviation between the test start point and the reference end point.

Embodiment 81: A computer-implemented method according to embodiment 80, wherein:
the extracted digital biomarker feature data is the clinical parameter.

Embodiment 82: A computer-implemented method according to embodiment 80, further comprising:
calculating the clinical parameter from the extracted digital biomarker feature data.

Embodiment 83: The computer-implemented method of any one of embodiments 80 to 82, wherein:
the reference start point is the same as the reference end point, and the reference path is a closed path.

Embodiment 84: The computer-implemented method of embodiment 83, wherein:
the closed path is a square, a circle or a figure-of-eight.

Embodiment 85: The computer-implemented method of any one of embodiments 80 to 82, wherein:
the reference start point is different from the reference end point, and the reference path is an open path; and
the digital biomarker feature data is the deviation between the test end point and the reference end point.

Embodiment 86: The computer-implemented method of embodiment 85, wherein:
the open path is a straight line, or a spiral.

Embodiment 87: The computer-implemented method of any one of embodiments 80 to 86, wherein:
the method comprises:
   receiving a plurality of inputs from the touchscreen display, each of the plurality of inputs indicative of a respective test path traced by a user attempting to trace the reference path on the display of the mobile device, the test path comprising: a test start point, a test end point, and a test path traced between the test start point and the test end point;
   extracting digital biomarker feature data from each of the plurality of received inputs, thereby generating a respective plurality of pieces of digital biomarker features data, each piece of digital biomarker feature data comprising:
      a deviation between the test end point and the reference end point for the respective received input;
      a deviation between the test start point and the reference start point; and/or
      a deviation between the test start point and the test end point for the respective input.

Embodiment 88: The computer-implemented method of embodiment 87, wherein:
the method comprises:
   deriving a statistical parameter from the plurality of pieces of digital biomarker feature data.

Embodiment 89: The computer-implemented method of embodiment 88, wherein:
the statistical parameter comprises one or more of:
   a mean;
   a standard deviation;
   a percentile;
   a kurtosis; and
   a median.

Embodiment 90: The computer-implemented method of any one of embodiments 87 to 89, wherein:
the plurality of received inputs includes:
   a first subset of received inputs, each indicative of a respective test path traced by a user attempting to trace the reference path on the touchscreen display of the mobile device using their dominant hand, the first subset of received inputs having a respective first subset of extracted pieces of digital biomarker data; and
   a second subset of receive inputs, each indicative of a respective test path traced by a user attempting to trace the reference path on the touchscreen display of the mobile device using their non-dominant hand, the second subset of received inputs having a respective second subset of extracted pieces of digital biomarker data;
the method further comprises:
   deriving a first statistical parameter corresponding to the first subset of extracted pieces of digital biomarker feature data;
   deriving a second statistical parameter corresponding to the second subset of extracted pieces of digital biomarker feature data; and
   calculating a handedness parameter by calculating the difference between the first statistical parameter and the second statistical parameter, and optionally dividing the difference by the first statistical parameter or the second statistical parameter.

Embodiment 91: The computer-implemented method of any one of embodiments 87 to 90, wherein:
the plurality of received inputs includes:
   a first subset of received inputs, each indicative of a respective test path traced by a user attempting to trace the reference path on the touchscreen display of the mobile device in a first direction, the first subset of received inputs having a respective first subset of extracted pieces of digital biomarker data; and
   a second subset of receive inputs, each indicative of a respective test path traced by a user attempting to trace the reference path on the touchscreen display of the mobile device in a second direction, opposite form the first direction, the second subset of received inputs having a respective second subset of extracted pieces of digital biomarker data;
the method further comprises:
   deriving a first statistical parameter corresponding to the first subset of extracted pieces of digital biomarker feature data;
   deriving a second statistical parameter corresponding to the second subset of extracted pieces of digital biomarker feature data; and
   calculating a directionality parameter by calculating the difference between the first statistical parameter and the second statistical parameter, and optionally dividing the difference by the first statistical parameter or the second statistical parameter.

Embodiment 92: The computer-implemented method of any one of embodiments 80 to 91, further comprising the steps of:
applying at least one analysis model to the digital biomarker feature data;
determining the clinical parameter based on the output of the at least one analysis model.

Embodiment 93: The computer-implemented method of embodiment 92, wherein:
the analysis model comprises a trained machine learning model.

Embodiment 94: The computer-implemented method of embodiment 93, wherein:
the analysis model is a regression model, and the trained machine learning model comprises one or more of the following algorithms:
   a deep learning algorithm;
   k nearest neighbours (kNN);
   linear regression;
   partial last-squares (PLS);
   random forest (RF); and
   extremely randomized trees (XT).

Embodiment 95: The computer implemented method of embodiment 93, wherein:
the analysis model is a classification model, and the trained machine learning model comprises one or more of the following algorithms:
   a deep learning algorithm;
   k nearest neighbours (kNN);
   support vector machines (SVM);
   linear discriminant analysis;
   quadratic discriminant analysis (QDA);
   naïve Bayes (NB);
   random forest (RF); and
   extremely randomized trees (XT).

Embodiment 96: The computer-implemented method of any one of embodiments 80 to 95, wherein:
the disease whose status is to be predicted is multiple sclerosis and the clinical parameter comprises an expanded disability status scale (EDSS) value,
the disease whose status is to be predicted is spinal muscular atrophy and the clinical parameter comprises a forced vital capacity (FVC) value, or
wherein the disease whose status is to be predicted is Huntington's disease and the clinical parameter comprises a total motor score (TMS) value.

Embodiment 97: The computer-implemented method of any one of embodiments 80 to 96, wherein:
the method further comprises determining the at least one analysis model, wherein determining the at least one analysis model comprises:
   (a) receiving input data via at least one communication interface, wherein the input data comprises a set of historical digital biomarker feature data, wherein the set historical digital biomarker feature data comprises a plurality of measured values indicative of the disease status to be predicted;
   (b) determining at least one training data set and at least one test data set from the input data set;
   (c) determining the analysis model by training a machine learning model comprising at least one algorithm with the training data set;
   (d) predicting the clinical parameter of the test data set using the determined analysis model;
   (e) determining performance of the determined analysis model based on the predicted clinical parameter and a true value of the clinical parameter of the test data set.

Embodiment 98: The computer-implemented method of embodiment 97, wherein:
in step (c) a plurality of analysis models is determined by training a plurality of machine learning models with the training data set, wherein the machine learning models are distinguished by their algorithm, wherein in step d) a plurality of clinical parameters is predicted on the test data set using the determined analysis models, and
wherein in step (e) the performance of each of the determined analysis models is determined based on the predicted clinical parameter and the true value of the clinical parameter of the test data set, wherein the method further comprises determining the analysis model having the best performance.

Embodiment 99: A system for quantitatively determining a clinical parameter indicative of a status or progression of a disease, the system including:
a mobile device having a touchscreen display, a user input interface, and a first processing unit; and
a second processing unit;
wherein:
   the mobile device is configured to provide a distal motor test to a user thereof, wherein providing the distal motor test comprises:
   the first processing unit causing the touchscreen display of the mobile device to display an image comprising: a target start point, a target end point, and indication of a target path to be traced between the start point and the end point;
the user input interface is configured to receive from the touchscreen display, an input indicative of a test path traced by a user attempting to trace the target path on the display of the mobile device, the test path comprising: a test start point, a test end point, and a test path traced between the test start point and the test end point;
the first processing unit or the second processing unit is configured to extract digital biomarker feature data from the received input, the digital biomarker feature data comprising:
a deviation between the test end point and the target end point; and/or
a deviation between the test start point and the test end point.

Embodiment 100: The system of embodiment 99, wherein:
the extracted digital biomarker feature data is the clinical parameter.

Embodiment 101: The system of embodiment 99, wherein:
the first processing unit or the second processing unit is configured to calculate the clinical parameter from the extracted digital biomarker feature data.

Embodiment 102: The system of any one of embodiments 99 to 101, wherein:
the target start point is the same as the target end point, and the target path is a closed path.

Embodiment 103: The system of embodiment 102, wherein:
the closed path is a square, a circle or a figure-of-eight.

Embodiment 104: The system of embodiment any one of embodiments 99 to 101, wherein:
the target start point is different from the target end point, and the target path is an open path; and
the digital biomarker feature data is the deviation between the test end point and the target end point.

Embodiment 105: The system of embodiment 104, wherein:
the open path is a straight line, or a spiral.

Embodiment 106: The system of any one of embodiments 99 to 105, wherein:
the user input interface is configured to receive a plurality of inputs from the touchscreen display, each of the plurality of inputs indicative of a respective test path traced by a user attempting to trace the reference path on the display of the mobile device, the test path comprising: a test start point, a test end point, and a test path traced between the test start point and the test end point; and
the first processing unit or the second processing unit is configured to extract digital biomarker feature data from each of the plurality of received inputs, thereby generating a respective plurality of pieces of digital biomarker features data, each piece of digital biomarker feature data comprising:
   a deviation between the test end point and the reference end point for the respective received input;
   a deviation between the test start point and the reference start point; and/or
   a deviation between the test start point and the test end point for the respective input.

Embodiment 107: The system of embodiment 106, wherein:
the first processing unit or the second processing unit is further configured to derive a statistical parameter from the plurality of pieces of digital biomarker feature data.

Embodiment 108: The system of embodiment 107, wherein:
the statistical parameter comprises one or more of:
   a mean;
   a standard deviation;
   a percentile;
   a kurtosis; and
   a median.

Embodiment 109: The system of any one of embodiments 106 to 108, wherein:
the plurality of received inputs includes:
   a first subset of received inputs, each indicative of a respective test path traced by a user attempting to trace the reference path on the touchscreen display of the mobile device using their dominant hand, the first subset of received inputs having a respective first subset of extracted pieces of digital biomarker data; and
   a second subset of receive inputs, each indicative of a respective test path traced by a user attempting to trace the reference path on the touchscreen display of the mobile device using their non-dominant hand, the second subset of received inputs having a respective second subset of extracted pieces of digital biomarker data; and
the first processing unit or the second processing unit is configured to:
   derive a first statistical parameter corresponding to the first subset of extracted pieces of digital biomarker feature data;
   derive a second statistical parameter corresponding to the second subset of extracted pieces of digital biomarker feature data; and
   calculate a handedness parameter by calculating the difference between the first statistical parameter and the second statistical parameter, and optionally dividing the difference by the first statistical parameter or the second statistical parameter.

Embodiment 110: The system of any one of embodiments 106 to 109, wherein:
the plurality of received inputs includes:
   a first subset of received inputs, each indicative of a respective test path traced by a user attempting to trace the reference path on the touchscreen display of the mobile device in a first direction, the first subset of received inputs having a respective first subset of extracted pieces of digital biomarker data; and
   a second subset of receive inputs, each indicative of a respective test path traced by a user attempting to trace the reference path on the touchscreen display of the mobile device in a second direction, opposite form the first direction, the second subset of received inputs having a respective second subset of extracted pieces of digital biomarker data;
the first processing unit or the second processing unit is configured to:
   derive a first statistical parameter corresponding to the first subset of extracted pieces of digital biomarker feature data;
   derive a second statistical parameter corresponding to the second subset of extracted pieces of digital biomarker feature data; and
   calculate a directionality parameter by calculating the difference between the first statistical parameter and the second statistical parameter, and optionally dividing the difference by the first statistical parameter or the second statistical parameter.

Embodiment 111: The system of any one of embodiments 99 to 110, wherein:
the second processing unit is configured to apply at least one analysis model to the digital biomarker feature data or a statistical parameter derived from the digital biomarker feature data, and to predict a value of the at least one clinical parameter based on an output of the at least one analysis model.

Embodiment 112: The system of embodiment 111, wherein:
the analysis model comprises a trained machine learning model.

Embodiment 113: The system of embodiment 112, wherein:
the analysis model is a regression model, and the trained machine learning model comprises one or more of the following algorithms:
   a deep learning algorithm;
   k nearest neighbours (kNN);
   linear regression;
   partial last-squares (PLS);
   random forest (RF); and
   extremely randomized trees (XT).

Embodiment 114: The system of embodiment 112, wherein:
the analysis model is a classification model, and the trained machine learning model comprises one or more of the following algorithms:
   a deep learning algorithm;
   k nearest neighbours (kNN);
   support vector machines (SVM);
   linear discriminant analysis;
   quadratic discriminant analysis (QDA);
   naïve Bayes (NB);
   random forest (RF); and
   extremely randomized trees (XT).

Embodiment 115: The system of any one of embodiments 99 to 114, wherein:
the disease whose status is to be predicted is multiple sclerosis and the clinical parameter comprises an expanded disability status scale (EDSS) value,
the disease whose status is to be predicted is spinal muscular atrophy and the clinical parameter comprises a forced vital capacity (FVC) value, or
wherein the disease whose status is to be predicted is Huntington's disease and the clinical parameter comprises a total motor score (TMS) value.

Embodiment 116: The system of any one of embodiments 99 to 115, wherein:
the first processing unit and the second processing unit are the same processing unit.

Embodiment 117: The system of any one of embodiments 99 to 115, wherein:
the first processing unit is separate from the second processing unit.

Embodiment 118: The system of any one of embodiments 99 to 117, further comprising a machine learning system for determining the at least one analysis model for predicting the at least one clinical parameter indicative of a disease status, the machine learning system comprising:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the clinical parameter of the test data set using the determined analysis model, wherein the processing unit is configured for determining performance of the determined analysis model based on the predicted clinical parameter and a true value of the clinical parameter of the test data set, wherein the processing unit is the first processing unit or the second processing unit.

Embodiment 119: A computer-implemented method comprising one, two, or all of:
the steps of any one of embodiments 1 to 38;
the steps of embodiment 78; and
the steps of any one of embodiments 80 to 98.

Embodiment 120: A system comprising one, two, or all of:
the system of any one of embodiments 39 to 77;
the system of embodiment 79; and
the system of any one of embodiments 99 to 118.

Prediction of a Status or Progression of a Disease

The above disclosure relates primarily to the determination of a clinical parameter which is indicative of a status or progression of a disease. However, in some cases, the invention may provide a computer-implemented method of determining a status or progression of a disease, the computer-implemented method comprising: providing a distal motor test to a user of a mobile device, the mobile device having a touchscreen display, wherein providing the distal motor test to the user of the mobile device comprises: causing the touchscreen display of the mobile device to display an image comprising: a reference start point, a reference end point, and indication of a reference path to be traced between the start point and the end point; receiving an input from the touchscreen display of the mobile device, the input indicative of a test path traced by a user attempting to trace the reference path on the display of the mobile device, the test path comprising: a test start point, a test end point, and a test path traced between the test start point and the test end point; and extracting digital biomarker feature data from the received input, the digital biomarker feature data comprising: a deviation between the test end point and the reference end point; a deviation between the test start point and the reference start point; and/or a deviation between the test start point and the reference end point; and wherein: the extracted digital biomarker feature data is the clinical parameter; or the method further comprises calculating the clinical parameter from the extracted biomarker feature data; and determining the status or progression of the disease based on the determined clinical parameter.

Equivalently, a further aspect of the invention provides a system for determining a status or progression of a disease, the system comprising; a mobile device having a touchscreen display, a user input interface, and a first processing unit; and a second processing unit; wherein: the mobile device is configured to provide a distal motor test to a user thereof, wherein providing the distal motor test comprises: the first processing unit causing the touchscreen display of the mobile device to display an image comprising: a reference start point, a reference end point, and indication of a reference path to be traced between the start point and the end point; the user input interface is configured to receive from the touchscreen display, an input indicative of a test path traced by a user attempting to trace the reference path on the display of the mobile device, the test path comprising: a test start point, a test end point, and a test path traced between the test start point and the test end point; and the first processing unit or the second processing unit is configured to extract digital biomarker feature data from the received input, the digital biomarker feature data comprising: a deviation between the test end point and the reference end point; and/or a deviation between the test start point and the test end point; and wherein: the extracted digital biomarker feature data is the clinical parameter; or the first processing unit or the second processing unit is further configured to calculate the clinical parameter from the extract digital biomarker feature data; and the first processing unit or the second processing unit is configured to determine the status or progression of the disease based on the determined clinical parameter.

It should be explicitly appreciated that the features of the two aspects of the invention set out here may be combined with the features of any of the “embodiments” set out above, except where clearly incompatible, or where context dictates otherwise. The features of these two aspects of the invention may also be combined with any of the subsequent disclosure.

Additional Related Aspects of the Disclosure

In a related aspect of the disclosure, a machine learning system for determining at least one analysis model for predicting at least one target variable indicative of a disease status is proposed. The machine learning system comprises:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the target variable on the test data set using the determined analysis model, wherein the processing unit is configured for determining performance of the determined analysis model based on the predicted target variable and a true value of the target variable of the test data set.

The term “machine learning” as used herein is a broad term and is to be given its ordinary and customary meaning to a person of ordinary skill in the art and is not to be limited to a special or customized meaning. The term specifically may refer, without limitation, to a method of using artificial intelligence (AI) for automatically model building of analytical models. The term “machine learning system” as used herein is a broad term and is to be given its ordinary and customary meaning to a person of ordinary skill in the art and is not to be limited to a special or customized meaning. The term specifically may refer, without limitation, to a system comprising at least one processing unit such as a processor, microprocessor, or computer system configured for machine learning, in particular for executing a logic in a given algorithm. The machine learning system may be configured for performing and/or executing at least one machine learning algorithm, wherein the machine learning algorithm is configured for building the at least one analysis model based on the training data.

The term “analysis model” as used herein is a broad term and is to be given its ordinary and customary meaning to a person of ordinary skill in the art and is not to be limited to a special or customized meaning. The term specifically may refer, without limitation, to a mathematical model configured for predicting at least one target variable for at least one state variable. The analysis model may be a regression model or a classification model. The term “regression model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a numerical value within a range. The term “classification model” as used herein is a broad term and is to be given its ordinary and customary meaning to a person of ordinary skill in the art and is not to be limited to a special or customized meaning. The term specifically may refer, without limitation, to an analysis model comprising at least one supervised learning algorithm having as output a classifier such as “μl” or “healthy”.

The term “target variable” as used herein is a broad term and is to be given its ordinary and customary meaning to a person of ordinary skill in the art and is not to be limited to a special or customized meaning. The term specifically may refer, without limitation, to a clinical value which is to be predicted. The target variable value which is to be predicted may dependent on the disease whose presence or status is to be predicted. The target variable may be either numerical or categorical. For example, the target variable may be categorical and may be “positive” in case of presence of disease or “negative” in case of absence of the disease.

The target variable may be numerical such as at least one value and/or scale value.

For example, the disease whose status is to be predicted is multiple sclerosis. The term “multiple sclerosis (MS)” as used herein relates to disease of the central nervous system (CNS) that typically causes prolonged and severe disability in a subject suffering therefrom. There are four standardized subtype definitions of MS which are also encompassed by the term as used in accordance with the present invention: relapsing-remitting, secondary progressive, primary progressive and progressive relapsing. The term relapsing forms of MS is also used and encompasses relapsing-remitting and secondary progressive MS with superimposed relapses. The relapsing-remitting subtype is characterized by unpredictable relapses followed by periods of months to years of remission with no new signs of clinical disease activity. Deficits suffered during attacks (active status) may either resolve or leave sequelae. This describes the initial course of 85 to 90% of subjects suffering from MS. Secondary progressive MS describes those with initial relapsing-remitting MS, who then begin to have progressive neurological decline between acute attacks without any definite periods of remission. Occasional relapses and minor remissions may appear. The median time between disease onset and conversion from relapsing remitting to secondary progressive MS is about 19 years. The primary progressive subtype describes about 10 to 15% of subjects who never have remission after their initial MS symptoms. It is characterized by progressive of disability from onset, with no, or only occasional and minor, remissions and improvements. The age of onset for the primary progressive subtype is later than other subtypes. Progressive relapsing MS describes those subjects who, from onset, have a steady neurological decline but also suffer clear superimposed attacks. It is now accepted that this latter progressive relapsing phenotype is a variant of primary progressive MS (PPMS) and diagnosis of PPMS according to McDonald 2010 criteria includes the progressive relapsing variant.

Symptoms associated with MS include changes in sensation (hypoesthesia and par-aesthesia), muscle weakness, muscle spasms, difficulty in moving, difficulties with co-ordination and balance (ataxia), problems in speech (dysarthria) or swallowing (dysphagia), visual problems (nystagmus, optic neuritis and reduced visual acuity, or diplopia), fatigue, acute or chronic pain, bladder, sexual and bowel difficulties. Cognitive impairment of varying degrees as well as emotional symptoms of depression or unstable mood are also frequent symptoms. The main clinical measure of disability progression and symptom severity is the Expanded Disability Status Scale (EDSS). Further symptoms of MS are well known in the art and are described in the standard text books of medicine and neurology.

The term “progressing MS” as used herein refers to a condition, where the disease and/or one or more of its symptoms get worse over time. Typically, the progression is accompanied by the appearance of active statuses. The said progression may occur in all subtypes of the disease. However, typically “progressing MS” shall be determined in accordance with the present invention in subjects suffering from relapsing-remitting MS.

Determining status of multiple sclerosis, generally comprises assessing at least one symptom associated with multiple sclerosis selected from a group consisting of: impaired fine motor abilities, pins an needs, numbness in the fingers, fatigue and changes to diurnal rhythms, gait problems and walking difficulty, cognitive impairment including problems with processing speed. Disability in multiple sclerosis may be quantified according to the expanded disability status scale (EDSS) as described in Kurtzke JF, “Rating neurologic impairment in multiple sclerosis: an expanded disability status scale (EDSS)”, November 1983, Neurology. 33 (11): 1444-52. doi:10.1212/VVNL.33.11.1444. PM ID 6685237. The target variable may be an EDSS value.

The term “expanded disability status scale (EDSS)” as used herein, thus, refers to a score based on quantitative assessment of the disabilities in subjects suffering from MS (Krutzke 1983). The EDSS is based on a neurological examination by a clinician. The EDSS quantifies disability in eight functional systems by assigning a Functional System Score (FSS) in each of these functional systems. The functional systems are the pyramidal system, the cerebellar system, the brainstem system, the sensory system, the bowel and bladder system, the visual system, the cerebral system and other (remaining) systems. EDSS steps 1.0 to 4.5 refer to subjects suffering from MS who are fully ambulatory, EDSS steps 5.0 to 9.5 characterize those with impairment to ambulation.

The clinical meaning of each possible result is the following:
0.0: Normal Neurological Exam
1.0: No disability, minimal signs in 1 FS
1.5: No disability, minimal signs in more than 1 FS
2.0: Minimal disability in 1 FS
2.5: Mild disability in 1 or Minimal disability in 2 FS
3.0: Moderate disability in 1 FS or mild disability in 3-4 FS, though fully ambulatory
3.5: Fully ambulatory but with moderate disability in 1 FS and mild disability in 1 or 2 FS; or moderate disability in 2 FS; or mild disability in 5 FS
4.0: Fully ambulatory without aid, up and about 12 hrs a day despite relatively severe disability. Able to walk without aid 500 meters
4.5: Fully ambulatory without aid, up and about much of day, able to work a full day, may otherwise have some limitations of full activity or require minimal assistance. Relatively severe disability. Able to walk without aid 300 meters
5.0: Ambulatory without aid for about 200 meters. Disability impairs full daily activities
5.5: Ambulatory for 100 meters, disability precludes full daily activities
6.0: Intermittent or unilateral constant assistance (cane, crutch or brace) required to walk 100 meters with or without resting
6.5: Constant bilateral support (cane, crutch or braces) required to walk 20 meters without resting
7.0: Unable to walk beyond 5 meters even with aid, essentially restricted to wheelchair, wheels self, transfers alone; active in wheelchair about 12 hours a day
7.5: Unable to take more than a few steps, restricted to wheelchair, may need aid to transfer; wheels self, but may require motorized chair for full day's activities
8.0: Essentially restricted to bed, chair, or wheelchair, but may be out of bed much of day; retains self-care functions, generally effective use of arms
8.5: Essentially restricted to bed much of day, some effective use of arms, retains some self-care functions
9.0: Helpless bed patient, can communicate and eat
9.5: Unable to communicate effectively or eat/swallow
10.0: Death due to MS

For example, the disease whose status is to be predicted is spinal muscular atrophy.

The term “spinal muscular atrophy (SMA)” as used herein relates to a neuromuscular disease which is characterized by the loss of motor neuron function, typically, in the spinal chord. As a consequence of the loss of motor neuron function, typically, muscle atrophy occurs resulting in an early dead of the affected subjects. The disease is caused by an inherited genetic defect in the SMN1 gene. The SMN protein encoded by said gene is required for motor neuron survival. The disease is inherited in an autosomal recessive manner.

Symptoms associated with SMA include areflexia, in particular, of the extremities, muscle weakness and poor muscle tone, difficulties in completing developmental phases in childhood, as a consequence of weakness of respiratory muscles, breathing problems occurs as well as secretion accumulation in the lung, as well as difficulties in sucking, swallowing and feeding/eating. Four different types of SMA are known.

The infantile SMA or SMA1 (Werdnig-Hoffmann disease) is a severe form that manifests in the first months of life, usually with a quick and unexpected onset (“floppy baby syndrome”). A rapid motor neuron death causes inefficiency of the major body organs, in particular, of the respiratory system, and pneumonia-induced respiratory failure is the most frequent cause of death. Unless placed on mechanical ventilation, babies diagnosed with SMA1 do not generally live past two years of age, with death occurring as early as within weeks in the most severe cases, sometimes termed SMA0. With proper respiratory support, those with milder SMA1 phenotypes accounting for around 10% of SMA1 cases are known to live into adolescence and adulthood.

The intermediate SMA or SMA2 (Dubowitz disease) affects children who are never able to stand and walk but who are able to maintain a sitting position at least some time in their life. The onset of weakness is usually noticed some time between 6 and 18 months. The progress is known to vary. Some people gradually grow weaker over time while others through careful maintenance avoid any progression. Scoliosis may be present in these children, and correction with a brace may help improve respiration. Muscles are weakened, and the respiratory system is a major concern. Life expectancy is somewhat reduced but most people with SMA2 live well into adulthood.

The juvenile SMA or SMA3 (Kugelberg-Welander disease) manifests, typically, after 12 months of age and describes people with SMA3 who are able to walk without support at some time, although many later lose this ability. Respiratory involvement is less noticeable, and life expectancy is normal or near normal.

The adult SMA or SMA4 manifests, usually, after the third decade of life with gradual weakening of muscles that affects proximal muscles of the extremities frequently requiring the person to use a wheelchair for mobility. Other complications are rare, and life expectancy is unaffected.

Typically, SMA in accordance with the present invention is SMA1 (Werdnig-Hoffmann disease), SMA2 (Dubowitz disease), SMA3 (Kugelberg-Welander diseases) or SMA4

SMA is typically diagnosed by the presence of the hypotonia and the absence of reflexes. Both can be measured by standard techniques by the clinician in a hospital including electromyography. Sometimes, serum creatine kinase may be increased as a biochemical parameter. Moreover, genetic testing is also possible, in particular, as prenatal diagnostics or carrier screening. Moreover, a critical parameter in SMA management is the function of the respiratory system. The function of the respiratory system can be, typically, determined by measuring the forced vital capacity of the subject which will be indicative for the degree of impairment of the respiratory system as a consequence of SMA.

The term “forced vital capacity (FVC)” as used herein refers to is the volume in liters of air that can forcibly be blown out after full inspiration by a subject. It is, typically, determined by spirometry in a hospital or at a doctor's residency using spirometric devices.

Determining status of spinal muscular atrophy, generally comprises assessing at least one symptom associated with spinal muscular atrophy selected from a group consisting of: hypotonia and muscle weakness, fatigue and changes to diurnal rhythms. A measure for status of spinal muscular atrophy may be the Forced vital capacity (FVC). The FVC may be a quantitative measure for volume of air that can forcibly be blown out after full inspiration, measured in liters, see https://en.wikipedia.org/wiki/Spirometry. The target variable may be a FVC value.

For example, the disease whose status is to be predicted is Huntington's disease.

The term “Huntington's Disease (HD)” as used herein relates to an inherited neurological disorder accompanied by neuronal cell death in the central nervous system. Most prominently, the basal ganglia are affected by cell death. There are also further areas of the brain involved such as substantia nigra, cerebral cortex, hippocampus and the purkinje cells. All regions, typically, play a role in movement and behavioral control. The disease is caused by genetic mutations in the gene encoding Huntingtin. Huntingtin is a protein involved in various cellular functions and interacts with over 100 other proteins. The mutated Huntingtin appears to be cytotoxic for certain neuronal cell types. Mutated Huntingtin is characterized by a poly glutamine region caused by a trinucleotide repeat in the Huntingtin gene. A repeat of more than 36 glutamine residues in the poly glutamine region of the protein results in the disease causing Huntingtin protein.

The symptoms of the disease most commonly become noticeable in the mid-age, but can begin at any age from infancy to the elderly. In early stages, symptoms involve subtle changes in personality, cognition, and physical skills. The physical symptoms are usually the first to be noticed, as cognitive and behavioral symptoms are generally not severe enough to be recognized on their own at said early stages. Almost everyone with HD eventually exhibits similar physical symptoms, but the onset, progression and extent of cognitive and behavioral symptoms vary significantly between individuals. The most characteristic initial physical symptoms are jerky, random, and uncontrollable movements called chorea. Chorea may be initially exhibited as general restlessness, small unintentionally initiated or uncompleted motions, lack of coordination, or slowed saccadic eye movements. These minor motor abnormalities usually precede more obvious signs of motor dysfunction by at least three years. The clear appearance of symptoms such as rigidity, writhing motions or abnormal posturing appear as the disorder progresses. These are signs that the system in the brain that is responsible for movement has been affected. Psychomotor functions become increasingly impaired, such that any action that requires muscle control is affected. Common consequences are physical instability, abnormal facial expression, and difficulties chewing, swallowing, and speaking. Consequently, eating difficulties and sleep disturbances are also accompanying the disease. Cognitive abilities are also impaired in a progressive manner. Impaired are executive functions, cognitive flexibility, abstract thinking, rule acquisition, and proper action/reaction capabilities. In more pronounced stages, memory deficits tend to appear including short-term memory deficits to long-term memory difficulties. Cognitive problems worsen over time and will ultimately turn into dementia. Psychiatric complications accompanying HD are anxiety, depression, a reduced display of emotions (blunted affect), egocentrism, aggression, and compulsive behavior, the latter of which can cause or worsen addictions, including alcoholism, gambling, and hypersexuality.

There is no cure for HD. There are supportive measurements in disease management depending on the symptoms to be addressed. Moreover, a number of drugs are used to ameliorate the disease, its progression or the symptoms accompanying it. Tetrabenazine is approved for treatment of HD, include neuroleptics and benzodiazepines are used as drugs that help to reduce chorea, amantadine or remacemide are still under investigation but have shown preliminary positive results. Hypokinesia and rigidity, especially in juvenile cases, can be treated with antiparkinsonian drugs, and myoclonic hyperkinesia can be treated with valproic acid. Ethyl-eicosapentoic acid was found to enhance the motor symptoms of patients, however, its long-term effects need to be revealed.

The disease can be diagnosed by genetic testing. Moreover, the severity of the disease can be staged according to Unified Huntington's Disease Rating Scale (UHDRS). This scale system addresses four components, i.e. the motor function, the cognition, behavior and functional abilities. The motor function assessment includes assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking and can be summarized as total motor score (TMS). The motoric functions must be investigated and judged by a medical practitioner.

Determining status of Huntington's disease generally comprises assessing at least one symptom associated with Huntington's disease selected from a group consisting of: Psychomotor slowing, chorea (jerking, writhing), progressive dysarthria, rigidity and dystonia, social withdrawal, progressive cognitive impairment of processing speed, attention, planning, visual-spatial processing, learning (though intact recall), fatigue and changes to diurnal rhythms. A measure for status of is a total motor score (TMS). The target variable may be a total motor score (TMS) value. The term “total motor score (TMS)” as used herein, thus, refers to a score based on assessment of ocular pursuit, saccade initiation, saccade velocity, dysarthria, tongue protrusion, maximal dystonia, maximal chorea, retropulsion pull test, finger taps, pronate/supinate hands, luria, rigidity arms, bradykinesia body, gait, and tandem walking.

The term “state variable” as used herein is a broad term and is to be given its ordinary and customary meaning to a person of ordinary skill in the art and is not to be limited to a special or customized meaning. The term specifically may refer, without limitation, to an input variable which can be filled in the prediction model such as data derived by medical examination and/or self-examination by a subject. The state variable may be determined in at least one active test and/or in at least one passive monitoring. For example, the state variable may be determined in an active test such as at least one cognition test and/or at least one hand motor function test and/or or at least one mobility test.

The term “subject” as used herein, typically, relates to mammals. The subject in accordance with the present invention may, typically, suffer from or shall be suspected to suffer from a disease, i.e. it may already show some or all of the negative symptoms associated with the said disease. In an embodiment of the invention said subject is a human.

The state variable may be determined by using at least one mobile device of the subject. The term “mobile device” as used herein is a broad term and is to be given its ordinary and customary meaning to a person of ordinary skill in the art and is not to be limited to a special or customized meaning. The term may specifically refer, without limitation, to a mobile electronics device, more specifically to a mobile communication device comprising at least one processor. The mobile device may specifically be a cell phone or smartphone. The mobile device may also refer to a tablet computer or any other type of portable computer. The mobile device may comprise a data acquisition unit which may be configured for data acquisition. The mobile device may be configured for detecting and/or measuring either quantitatively or qualitatively physical parameters and transform them into electronic signals such as for further processing and/or analysis. For this purpose, the mobile device may comprise at least one sensor. It will be understood that more than one sensor can be used in the mobile device, i.e. at least two, at least three, at least four, at least five, at least six, at least seven, at least eight, at least nine or at least ten or even more different sensors. The sensor may be at least one sensor selected from the group consisting of: at least one gyroscope, at least one magnetometer, at least one accelerometer, at least one proximity sensor, at least one thermometer, at least one pedometer, at least one fingerprint detector, at least one touch sensor, at least one voice recorder, at least one light sensor, at least one pressure sensor, at least one location data detector, at least one camera, at least one GPS, and the like. The mobile device may comprise the processor and at least one database as well as software which is tangibly embedded to said device and, when running on said device, carries out a method for data acquisition. The mobile device may comprise a user interface, such as a display and/or at least one key, e.g. for performing at least one task requested in the method for data acquisition.

The term “predicting” as used herein is a broad term and is to be given its ordinary and customary meaning to a person of ordinary skill in the art and is not to be limited to a special or customized meaning. The term specifically may refer, without limitation, to determining at least one numerical or categorical value indicative of the disease status for the at least one state variable. In particular, the state variable may be filled in the analysis as input and the analysis model may be configured for performing at least one analysis on the state variable for determining the at least one numerical or categorical value indicative of the disease status. The analysis may comprise using the at least one trained algorithm.

The term “determining at least one analysis model” as used herein is a broad term and is to be given its ordinary and customary meaning to a person of ordinary skill in the art and is not to be limited to a special or customized meaning. The term specifically may refer, without limitation, to building and/or creating the analysis model.

The term “disease status” as used herein is a broad term and is to be given its ordinary and customary meaning to a person of ordinary skill in the art and is not to be limited to a special or customized meaning. The term specifically may refer, without limitation, to health condition and/or medical condition and/or disease stage. For example, the disease status may be healthy or ill and/or presence or absence of disease. For example, the disease status may be a value relating to a scale indicative of disease stage. The term “indicative of a disease status” as used herein is a broad term and is to be given its ordinary and customary meaning to a person of ordinary skill in the art and is not to be limited to a special or customized meaning. The term specifically may refer, without limitation, to information directly relating to the disease status and/or to information indirectly relating to the disease status, e.g. information which need further analysis and/or processing for deriving the disease status. For example, the target variable may be a value which need to be compared to a table and/or lookup table for determine the disease status.

The term “communication interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. In particular, the communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information. The communication interface may specifically provide means for transferring or exchanging information. In particular, the communication interface may provide a data transfer connection, e.g. Bluetooth, NFC, inductive coupling or the like. As an example, the communication interface may be or may comprise at least one port comprising one or more of a network or internet port, a USB-port and a disk drive. The communication interface may be at least one web interface.

The term “input data” as used herein is a broad term and is to be given its ordinary and customary meaning to a person of ordinary skill in the art and is not to be limited to a special or customized meaning. The term specifically may refer, without limitation, to experimental data used for model building. The input data comprises the set of historical digital biomarker feature data. The term “biomarker” as used herein is a broad term and is to be given its ordinary and customary meaning to a person of ordinary skill in the art and is not to be limited to a special or customized meaning. The term specifically may refer, without limitation, to a measurable characteristic of a biological state and/or biological condition. The term “feature” as used herein is a broad term and is to be given its ordinary and customary meaning to a person of ordinary skill in the art and is not to be limited to a special or customized meaning. The term specifically may refer, without limitation, to a measurable property and/or characteristic of a symptom of the disease on which the prediction is based. In particular, all features from all tests may be considered and the optimal set of features for each prediction is determined. Thus, all features may be considered for each disease. The term “digital biomarker feature data” as used herein is a broad term and is to be given its ordinary and customary meaning to a person of ordinary skill in the art and is not to be limited to a special or customized meaning. The term specifically may refer, without limitation, to experimental data determined by at least one digital device such as by a mobile device which comprises a plurality of different measurement values per subject relating to symptoms of the disease. The digital biomarker feature data may be determined by using at least one mobile device. With respect to the mobile device and determining of digital biomarker feature data with the mobile device reference is made to the description of the determination of the state variable with the mobile device above. The set of historical digital biomarker feature data comprises a plurality of measured values per subject indicative of the disease status to be predicted. The term “historical” as used herein is a broad term and is to be given its ordinary and customary meaning to a person of ordinary skill in the art and is not to be limited to a special or customized meaning. The term specifically may refer, without limitation, to the fact that the digital biomarker feature data was determined and/or collected before model building such as during at least one test study. For example, for model building for predicting at least one target indicative of multiple sclerosis the digital biomarker feature data may be data from Floodlight POC study. For example, for model building for predicting at least one target indicative of spinal muscular atrophy the digital biomarker feature data may be data from OLEOS study. For example, for model building for predicting at least one target indicative of Huntington's disease the digital biomarker feature data may be data from HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The term “target data” as used herein is a broad term and is to be given its ordinary and customary meaning to a person of ordinary skill in the art and is not to be limited to a special or customized meaning. The term specifically may refer, without limitation, to data comprising clinical values to predict, in particular one clinical value per subject. The target data may be either numerical or categorical. The clinical value may directly or indirectly refer to the status of the disease.

The processing unit may be configured for extracting features from the input data. The term “extracting features” as used herein is a broad term and is to be given its ordinary and customary meaning to a person of ordinary skill in the art and is not to be limited to a special or customized meaning. The term specifically may refer, without limitation, to at least one process of determining and/or deriving features from the input data. Specifically, the features may be pre-defined, and a subset of features may be selected from an entire set of possible features. The extracting of features may comprise one or more of data aggregation, data reduction, data transformation and the like. The processing unit may be configured for ranking the features. The term “ranking features” as used herein is a broad term and is to be given its ordinary and customary meaning to a person of ordinary skill in the art and is not to be limited to a special or customized meaning. The term specifically may refer, without limitation, to assigning a rank, in particular a weight, to each of the features depending on predefined criteria. For example, the features may be ranked with respect to their relevance, i.e. with respect to correlation with the target variable, and/or the features may be ranked with respect to redundancy, i.e. with respect to correlation between features. The processing unit may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2):185-205, PubMed

PMI D:15852500. The feature selection and ranking may be performed by using a modified method compared to the method described in Ding et al.. The maximum correlation coefficient may be used rather than the mean correlation coefficient and an addition transformation may be applied to it. In case of a regression model as analysis model the transformation the value of the mean correlation coefficient may be raised to the 5^{th} power. In case of a classification model as analysis model the value of the mean correlation coefficient may be multiplied by 10.

The term “model unit” as used herein is a broad term and is to be given its ordinary and customary meaning to a person of ordinary skill in the art and is not to be limited to a special or customized meaning. The term specifically may refer, without limitation, to at least one data storage and/or storage unit configured for storing at least one machine learning model. The term “machine learning model” as used herein is a broad term and is to be given its ordinary and customary meaning to a person of ordinary skill in the art and is not to be limited to a special or customized meaning. The term specifically may refer, without limitation, to at least one trainable algorithm. The model unit may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing operations of a computer or system, and/or, generally, to a device which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may be configured for machine learning. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The processing unit may be configured for pre-processing the input data. The pre-processing may comprise at least one filtering process for input data fulfilling at least one quality criterion. For example, the input data may be filtered to remove missing variables. For example, the pre-processing may comprise excluding data from subjects with less than a pre-defined minimum number of observations.

The term “training data set” as used herein is a broad term and is to be given its ordinary and customary meaning to a person of ordinary skill in the art and is not to be limited to a special or customized meaning. The term specifically may refer, without limitation, to a subset of the input data used for training the machine learning model. The term “test data set” as used herein is a broad term and is to be given its ordinary and customary meaning to a person of ordinary skill in the art and is not to be limited to a special or customized meaning. The term specifically may refer, without limitation, to another subset of the input data used for testing the trained machine learning model. The training data set may comprise a plurality of training data sets. In particular, the training data set comprises a training data set per subject of the input data. The test data set may comprise a plurality of test data sets. In particular, the test data set comprises a test data set per subject of the input data. The processing unit may be configured for generating and/or creating per subject of the input data a training data set and a test data set, wherein the test data set per subject may comprise data only of that subject, whereas the training data set for that subject comprises all other input data.

The processing unit may be configured for performing at least one data aggregation and/or data transformation on both of the training data set and the test data set for each subject. The transformation and feature ranking steps may be performed without splitting into training data set and test data set. This may allow to enable interference of e.g. important feature from the data.

The processing unit may be configured for one or more of at least one stabilizing transformation; at least one aggregation; and at least one normalization for the training data set and for the test data set.

For example, the processing unit may be configured for subject-wise data aggregation of both of the training data set and the test data set, wherein a mean value of the features is determined for each subject.

For example, the processing unit may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values >=0. The processing unit may be configured for transforming values of each feature using each of the variance transformation functions. The processing unit may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit may be configured for determining the best possible transformation, if any are better than the original values, on the training data set. The best possible transformation can be subsequently applied to the test data set.

For example, the processing unit may be configured for z-score transformation, wherein for each transformed feature the mean and standard deviations are determined on the training data set, wherein these values are used for z-score transformation on both the training data set and the test data set.

For example, the processing unit may be configured for performing three data transformation steps on both the training data set and the test data set, wherein the transformation steps comprise: 1. subject-wise data aggregation; 2. variance stabilization; 3. z-score transformation.

The processing unit may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit is configured for determining the analysis model by training the machine learning model with the training data set. The term “training the machine learning model” as used herein is a broad term and is to be given its ordinary and customary meaning to a person of ordinary skill in the art and is not to be limited to a special or customized meaning. The term specifically may refer, without limitation, to a process of determining parameters of the algorithm of machine learning model on the training data set. The training may comprise at least one optimization or tuning process, wherein a best parameter combination is determined. The training may be performed iteratively on the training data sets of different subjects. The processing unit may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyper-parameters.

In particular subsequent to the training of the machine learning model, the processing unit is configured for predicting the target variable on the test data set using the determined analysis model. The term “determined analysis model” as used herein is a broad term and is to be given its ordinary and customary meaning to a person of ordinary skill in the art and is not to be limited to a special or customized meaning. The term specifically may refer, without limitation, to the trained machine learning model. The processing unit may be configured for predicting the target variable for each subject based on the test data set of that subject using the determined analysis model. The processing unit may be configured for predicting the target variable for each subject on the respective training and test data sets using the analysis model. The processing unit may be configured for recording and/or storing both the predicted target variable per subject and the true value of the target variable per subject, for example, in at least one output file. The term “true value of the target variable” as used herein is a broad term and is to be given its ordinary and customary meaning to a person of ordinary skill in the art and is not to be limited to a special or customized meaning. The term specifically may refer, without limitation, to the real or actual value of the target variable of that subject, which may be determined from the target data of that subject.

The processing unit is configured for determining performance of the determined analysis model based on the predicted target variable and the true value of the target variable of the test data set. The term “performance” as used herein is a broad term and is to be given its ordinary and customary meaning to a person of ordinary skill in the art and is not to be limited to a special or customized meaning. The term specifically may refer, without limitation, to suitability of the determined analysis model for predicting the target variable. The performance may be characterized by deviations between predicted target variable and true value of the target variable. The machine learning system may comprises at least one output interface. The output interface may be designed identical to the communication interface and/or may be formed integral with the communication interface. The output interface may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit may comprise a plurality of machine learning models, wherein the machine learning models are distinguished by their algorithm. For example, for building a regression model the model unit may comprise the following algorithms k nearest neighbors (kNN), linear regression, partial last-squares (PLS), random forest (RF), and extremely randomized Trees (XT). For example, for building a classification model the model unit may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit may be configured for determining a analysis model for each of the machine learning models by training the respective machine learning model with the training data set and for predicting the target variables on the test data set using the determined analysis models.

The processing unit may be configured for determining performance of each of the determined analysis models based on the predicted target variables and the true value of the target variable of the test data set. In case of building a regression model, the output provided by the processing unit may comprise one or more of at least one scoring chart, at least one predictions plot, at least one correlations plot, and at least one residuals plot. The scoring chart may be a box plot depicting for each subject a mean absolute error from both the test and training data set and for each type of regressor, i.e. the algorithm which was used, and number of features selected. The predictions plot may show for each combination of regressor type and number of features, how well the predicted values of the target variable correlate with the true value, for both the test and the training data. The correlations plot may show the Spearman correlation coefficient between the predicted and true target variables, for each regressor type, as a function of the number of features included in the model. The residuals plot may show the correlation between the predicted target variable and the residual for each combination of regressor type and number of features, and for both the test and training data. The processing unit may be configured for determining the analysis model having the best performance, in particular based on the output.

In case of building a classification model, the output provided by the processing unit may comprise the scoring chart, showing in a box plot for each subject the mean F1 performance score, also denoted as F-score or F-measure, from both the test and training data and for each type of regressor and number of features selected. The processing unit may be configured for determining the analysis model having the best performance, in particular based on the output.

In a further related aspect of the disclosure, a computer implemented method for determining at least one analysis model for predicting at least one target variable indicative of a disease status is proposed. In the method a machine learning system according to the present invention is used. Thus, with respect to embodiments and definitions of the method reference is made to the description of the machine learning system above or as described in further detail below.

The method comprises the following method steps which, specifically, may be performed in the given order. Still, a different order is also possible. It is further possible to perform two or more of the method steps fully or partially simultaneously. Further, one or more or even all of the method steps may be performed once or may be performed repeatedly, such as repeated once or several times. Further, the method may comprise additional method steps which are not listed.

The method comprises the following steps:
a) receiving input data via at least one communication interface, wherein the input data comprises a set of historical digital biomarker feature data, wherein the set historical digital biomarker feature data comprises a plurality of measured values indicative of the disease status to be predicted;
at at least one processing unit:
b) determining at least one training data set and at least one test data set from the input data set;
c) determining the analysis model by training a machine learning model comprising at least one algorithm with the training data set;
d) predicting the target variable on the test data set using the determined analysis model;
e) determining performance of the determined analysis model based on the predicted target variable and a true value of the target variable of the test data set.

In step c) a plurality of analysis models may be determined by training a plurality of machine learning models with the training data set. The machine learning models may be distinguished by their algorithm. In step d) a plurality of target variables may be predicted on the test data set using the determined analysis models. In step e) the performance of each of the determined analysis models may be determined based on the predicted target variables and the true value of the target variable of the test data set. The method further may comprise determining the analysis model having the best performance.

Further disclosed and proposed herein is a computer program for determining at least one analysis model for predicting at least one target variable indicative of a disease status including computer-executable instructions for performing the method according to the present invention in one or more of the embodiments enclosed herein when the program is executed on a computer or computer network. Specifically, the computer program may be stored on a computer-readable data carrier and/or on a computer-readable storage medium. The computer program is configured to perform at least steps b) to e) of the method according to the present invention in one or more of the embodiments enclosed herein.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps b) to e) as indicated above may be performed by using a computer or a computer network, preferably by using a computer program.

Further disclosed and proposed herein is a computer program product having program code means, in order to perform the method according to the present invention in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In a further aspect of the present invention a use of a machine learning system according to according to one or more of the embodiments disclosed herein is proposed for predicting one or more of an expanded disability status scale (EDSS) value indicative of multiple sclerosis, a forced vital capacity (FVC) value indicative of spinal muscular atrophy, or a total motor score (TMS) value indicative of Huntington's disease.

The devices and methods according to the present invention have several advantages over known methods for predicting disease status. The use of a machine learning system may allow to analyze large amount of complex input data, such as data determined in several and large test studies, and allow to determine analysis models which allow delivering fast, reliable and accurate results.

Summarizing and without excluding further possible embodiments, the following additional embodiments may be envisaged, which may be combined with any of the previous embodiments:

Additional embodiment 1: A machine learning system for determining at least one analysis model for predicting at least one target variable indicative of a disease status comprising:
at least one communication interface configured for receiving input data, wherein the input data comprises a set of historical digital biomarker feature data, wherein the set of historical digital biomarker feature data comprises a plurality of measured values indicative of the disease status to be predicted;
at least one model unit comprising at least one machine learning model comprising at least one algorithm;
at least one processing unit, wherein the processing unit is configured for determining at least one training data set and at least one test data set from the input data set, wherein the processing unit is configured for determining the analysis model by training the machine learning model with the training data set, wherein the processing unit is configured for predicting the target variable on the test data set using the determined analysis model, wherein the processing unit is configured for determining performance of the determined analysis model based on the predicted target variable and a true value of the target variable of the test data set.

Additional embodiment 2: The machine learning system according to the preceding embodiment, wherein the analysis model is a regression model or a classification model.

Additional embodiment 3: The machine learning system according to the preceding embodiment, wherein the analysis model is a regression model, wherein the algorithm of the machine learning model is at least one algorithm selected from the group consisting of: k nearest neighbors (kNN); linear regression; partial last-squares (PLS); random forest (RF); and extremely randomized Trees (XT), or wherein the analysis model is a classification model, wherein the algorithm of the machine learning model is at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

Additional embodiment 4: The machine learning system according to any one of the preceding embodiments, wherein the model unit comprises a plurality of machine learning models, wherein the machine learning models are distinguished by their algorithm.

Additional embodiment 5: The machine learning system according to the preceding embodiment, wherein the processing unit is configured for determining a analysis model for each of the machine learning models by training the respective machine learning model with the training data set and for predicting the target variables on the test data set using the determined analysis models, wherein the processing unit is configured for determining performance of each of the determined analysis models based on the predicted target variables and the true value of the target variable of the test data set, wherein the processing unit is configured for determining the analysis model having the best performance.

Additional embodiment 6: The machine learning system according to any one of the preceding embodiments, wherein the target variable is a clinical value to be predicted, wherein the target variable is either numerical or categorical.

Additional embodiment 7: The machine learning system according to any one of the preceding embodiments, wherein the disease whose status is to be predicted is multiple sclerosis and the target variable is an expanded disability status scale (EDSS) value, or wherein the disease whose status is to be predicted is spinal muscular atrophy and the target variable is a forced vital capacity (FVC) value, or wherein the disease whose status is to be predicted is Huntington's disease and the target variable is a total motor score (TMS) value.

Additional embodiment 8: The machine learning system according to any one of the preceding embodiments, wherein the processing unit is configured for generating and/or creating per subject of the input data a training data set and a test data set, wherein the test data set comprises data of one subject, wherein the training data set comprises the other input data.

Additional embodiment 9: The machine learning system according to any one of the preceding embodiments, wherein the processing unit is configured for extracting features from the input data, wherein the processing unit is configured for ranking the features by using a maximum-relevance-minimum-redundancy technique.

Additional embodiment 10: The machine learning system according to the preceding embodiment, wherein the processing unit is configured for considering different numbers of features for determining the analysis model by training the machine learning model with the training data set.

Additional embodiment 11: The machine learning system according to any one of the preceding embodiments, wherein the processing unit is configured for pre-processing the input data, wherein the pre-processing comprises at least one filtering process for input data fulfilling at least one quality criterion.

Additional embodiment 12: The machine learning system according to any one of the preceding embodiments, wherein the processing unit is configured for performing one or more of at least one stabilizing transformation; at least one aggregation; and at least one normalization for the training data set and for the test data set.

Additional embodiment 13: The machine learning system according to any one of the preceding embodiments, wherein the machine learning system comprises at least one output interface, wherein the output interface is configured for providing at least one output, wherein the output comprises at least one information about the performance of the determined analysis model.

Additional embodiment 14: The machine learning system according to the preceding embodiment, wherein the information about the performance of the determined analysis model comprises one or more of at least one scoring chart, at least one predictions plot, at least one correlations plot, and at least one residuals plot.

Additional embodiment 15: A computer-implemented method for determining at least one analysis model for predicting at least one target variable indicative of a disease status, wherein in the method a machine learning system according to any one of the preceding embodiments is used, wherein the method comprises the following steps:
a) receiving input data via at least one communication interface, wherein the input data comprises a set of historical digital biomarker feature data, wherein the set historical digital biomarker feature data comprises a plurality of measured values indicative of the disease status to be predicted;
at at least one processing unit:
b) determining at least one training data set and at least one test data set from the input data set;
c) determining the analysis model by training a machine learning model comprising at least one algorithm with the training data set;
d) predicting the target variable on the test data set using the determined analysis model;
e) determining performance of the determined analysis model based on the predicted target variable and a true value of the target variable of the test data set.

Additional embodiment 16: The method according to the preceding embodiment, wherein in step c) a plurality of analysis models is determined by training a plurality of machine learning models with the training data set, wherein the machine learning models are distinguished by their algorithm, wherein in step d) a plurality of target variables is predicted on the test data set using the determined analysis models, wherein in step e) the performance of each of the determined analysis models is determined based on the predicted target variables and the true value of the target variable of the test data set, wherein the method further comprises determining the analysis model having the best performance.

Additional embodiment 17: Computer program for determining at least one analysis model for predicting at least one target variable indicative of a disease status, configured for causing a computer or computer network to fully or partially perform the method for determining at least one analysis model for predicting at least one target variable indicative of a disease status according to any one of the preceding embodiments referring to a method, when executed on the computer or computer network, wherein the computer program is configured to perform at least steps b) to e) of the method for determining at least one analysis model for predicting at least one target variable indicative of a disease status according to any one of the preceding embodiments referring to a method.

Additional embodiment 18: A computer-readable storage medium comprising instructions which, when executed by a computer or computer network cause to carry out at least steps b) to e) of the method according to any one of the preceding method embodiments.

Additional embodiment 19: Use of a machine learning system according to any one of the preceding embodiments referring to a machine learning system for determining an analysis model for predicting one or more of an expanded disability status scale (EDSS) value indicative of multiple sclerosis, a forced vital capacity (FVC) value indicative of spinal muscular atrophy, or a total motor score (TMS) value indicative of Huntington's disease.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these FIGS. refer to identical or functionally comparable elements.

In the drawings:

FIG. 1 shows an exemplary embodiment of a machine learning system according to the present invention;

FIG. 2 shows an exemplary embodiment of a computer-implemented method according to the present invention; and

FIGS. 3A to 3C show embodiments of correlations plots for assessment of performance of an analysis model.

FIG. 4 shows an example of a system which may be used to implement a method of the present invention.

FIG. 5A shows an example of a touchscreen display during a pinching test.

FIG. 5B shows an example of a touchscreen after a pinching test has been carried out, in order to illustrate some of the digital biomarker features which may be extracted.

FIGS. 6A to 6D show additional examples of pinching tests, illustrating various parameters.

FIG. 7 illustrates an example of a draw-a-shape test.

FIG. 8 illustrates an example of a draw-a-shape test.

FIG. 9 illustrates an example of a draw-a-shape test.

FIG. 10 illustrates an example of a draw-a-shape test.

FIG. 11 illustrates an end trace distance feature.

FIGS. 12A to 12C illustrate a begin-end trace distance feature.

FIGS. 13A to 13C illustrate a begin trace distance feature.

### DETAILED DESCRIPTION OF THE EMBODIMENTS

FIG. 1 shows highly schematically an embodiment of a machine learning system 110 for determining at least one analysis model for predicting at least one target variable indicative of a disease status.

The analysis model may be a mathematical model configured for predicting at least one target variable for at least one state variable. The analysis model may be a regression model or a classification model. The regression model may be an analysis model comprising at least one supervised learning algorithm having as output a numerical value within a range. The classification model may be an analysis model comprising at least one supervised learning algorithm having as output a classifier such as “ill” or “healthy”.

The target variable value which is to be predicted may dependent on the disease whose presence or status is to be predicted. The target variable may be either numerical or categorical. For example, the target variable may be categorical and may be “positive” in case of presence of disease or “negative” in case of absence of the disease. The disease status may be a health condition and/or a medical condition and/or a disease stage. For example, the disease status may be healthy or ill and/or presence or absence of disease. For example, the disease status may be a value relating to a scale indicative of disease stage. The target variable may be numerical such as at least one value and/or scale value. The target variable may directly relate to the disease status and/or may indirectly relate to the disease status. For example, the target variable may need further analysis and/or processing for deriving the disease status. For example, the target variable may be a value which need to be compared to a table and/or lookup table for determine the disease status.

The machine learning system 110 comprises at least one processing unit 112 such as a processor, microprocessor, or computer system configured for machine learning, in particular for executing a logic in a given algorithm. The machine learning system 110 may be configured for performing and/or executing at least one machine learning algorithm, wherein the machine learning algorithm is configured for building the at least one analysis model based on the training data. The processing unit 112 may comprise at least one processor. In particular, the processing unit 112 may be configured for processing basic instructions that drive the computer or system. As an example, the processing unit 112 may comprise at least one arithmetic logic unit (ALU), at least one floating-point unit (FPU), such as a math coprocessor or a numeric coprocessor, a plurality of registers and a memory, such as a cache memory. In particular, the processing unit 112 may be a multi-core processor. The processing unit 112 may be configured for machine learning. The processing unit 112 may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The machine learning system comprises at least one communication interface 114 configured for receiving input data. The communication interface 114 may be configured for transferring information from a computational device, e.g. a computer, such as to send or output information, e.g. onto another device. Additionally or alternatively, the communication interface 114 may be configured for transferring information onto a computational device, e.g. onto a computer, such as to receive information. The communication interface 114 may specifically provide means for transferring or exchanging information. In particular, the communication interface 114 may provide a data transfer connection, e.g. Bluetooth, NFC, inductive coupling or the like. As an example, the communication interface 114 may be or may comprise at least one port comprising one or more of a network or internet port, a USB-port and a disk drive. The communication interface 114 may be at least one web interface.

The input data comprises a set of historical digital biomarker feature data, wherein the set of historical digital biomarker feature data comprises a plurality of measured values indicative of the disease status to be predicted. The set of historical digital biomarker feature data comprises a plurality of measured values per subject indicative of the disease status to be predicted. For example, for model building for predicting at least one target indicative of multiple sclerosis the digital biomarker feature data may be data from Floodlight POC study. For example, for model building for predicting at least one target indicative of spinal muscular atrophy the digital biomarker feature data may be data from OLEOS study. For example, for model building for predicting at least one target indicative of Huntington's disease the digital biomarker feature data may be data from HD OLE study, ISIS 44319-CS2. The input data may be determined in at least one active test and/or in at least one passive monitoring. For example, the input data may be determined in an active test using at least one mobile device such as at least one cognition test and/or at least one hand motor function test and/or or at least one mobility test.

The input data further may comprise target data. The target data comprises clinical values to predict, in particular one clinical value per subject. The target data may be either numerical or categorical. The clinical value may directly or indirectly refer to the status of the disease.

The processing unit 112 may be configured for extracting features from the input data. The extracting of features may comprise one or more of data aggregation, data reduction, data transformation and the like. The processing unit 112 may be configured for ranking the features. For example, the features may be ranked with respect to their relevance, i.e. with respect to correlation with the target variable, and/or the features may be ranked with respect to redundancy, i.e. with respect to correlation between features. The processing unit 110 may be configured for ranking the features by using a maximum-relevance-minimum-redundancy technique. This method ranks all features using a trade-off between relevance and redundancy. Specifically, the feature selection and ranking may be performed as described in Ding C., Peng H. “Minimum redundancy feature selection from microarray gene expression data”, J Bioinform Comput Biol. 2005 April; 3 (2):185-205, PubMed PMI D:15852500. The feature selection and ranking may be performed by using a modified method compared to the method described in Ding et al.. The maximum correlation coefficient may be used rather than the mean correlation coefficient and an addition transformation may be applied to it. In case of a regression model as analysis model the transformation the value of the mean correlation coefficient may be raised to the 5th power. In case of a classification model as analysis model the value of the mean correlation coefficient may be multiplied by 10.

The machine learning system 110 comprises at least one model unit 116 comprising at least one machine learning model comprising at least one algorithm. The model unit 116 may comprise a plurality of machine learning models, e.g. different machine learning models for building the regression model and machine learning models for building the classification model. For example, the analysis model may be a regression model and the algorithm of the machine learning model may be at least one algorithm selected from the group consisting of: k nearest neighbors (kNN); linear regression; partial last-squares (PLS); random forest (RF); and extremely randomized Trees (XT). For example, the analysis model may be a classification model and the algorithm of the machine learning model may be at least one algorithm selected from the group consisting of: k nearest neighbors (kNN); support vector machines (SVM); linear discriminant analysis (LDA); quadratic discriminant analysis (QDA); naïve Bayes (NB); random forest (RF); and extremely randomized Trees (XT).

The processing unit 112 may be configured for pre-processing the input data. The pre-processing 112 may comprise at least one filtering process for input data fulfilling at least one quality criterion. For example, the input data may be filtered to remove missing variables. For example, the pre-processing may comprise excluding data from subjects with less than a pre-defined minimum number of observations.

The processing unit 112 is configured for determining at least one training data set and at least one test data set from the input data set. The training data set may comprise a plurality of training data sets. In particular, the training data set comprises a training data set per subject of the input data. The test data set may comprise a plurality of test data sets. In particular, the test data set comprises a test data set per subject of the input data. The processing unit 112 may be configured for generating and/or creating per subject of the input data a training data set and a test data set, wherein the test data set per subject may comprise data only of that subject, whereas the training data set for that subject comprises all other input data.

The processing unit 112 may be configured for performing at least one data aggregation and/or data transformation on both of the training data set and the test data set for each subject. The transformation and feature ranking steps may be performed without splitting into training data set and test data set. This may allow to enable interference of e.g. important feature from the data. The processing unit 112 may be configured for one or more of at least one stabilizing transformation; at least one aggregation; and at least one normalization for the training data set and for the test data set. For example, the processing unit 112 may be configured for subject-wise data aggregation of both of the training data set and the test data set, wherein a mean value of the features is determined for each subject. For example, the processing unit 112 may be configured for variance stabilization, wherein for each feature at least one variance stabilizing function is applied. The variance stabilizing function may be at least one function selected from the group consisting of: a logistic, which may be used if all values are greater 300 and no values are between 0 and 1; a logit, which may be used if all values are between 0 and 1, inclusive; a sigmoid; a log 10, which may be used if considered when all values >=0. The processing unit 112 may be configured for transforming values of each feature using each of the variance transformation functions. The processing unit 112 may be configured for evaluating each of the resulting distributions, including the original one, using a certain criterion. In case of a classification model as analysis model, i.e. when the target variable is discrete, said criterion may be to what extent the obtained values are able to separate the different classes. Specifically, the maximum of all class-wise mean silhouette values may be used for this end. In case of a regression model as analysis model, the criterion may be a mean absolute error obtained after regression of values, which were obtained by applying the variance stabilizing function, against the target variable. Using this selection criterion, processing unit 112 may be configured for determining the best possible transformation, if any are better than the original values, on the training data set. The best possible transformation can be subsequently applied to the test data set. For example, the processing unit 112 may be configured for z-score transformation, wherein for each transformed feature the mean and standard deviations are determined on the training data set, wherein these values are used for z-score transformation on both the training data set and the test data set. For example, the processing unit 112 may be configured for performing three data transformation steps on both the training data set and the test data set, wherein the transformation steps comprise: 1. subject-wise data aggregation; 2. variance stabilization; 3. z-score transformation. The processing unit 112 may be configured for determining and/or providing at least one output of the ranking and transformation steps. For example, the output of the ranking and transformation steps may comprise at least one diagnostics plots. The diagnostics plot may comprise at least one principal component analysis (PCA) plot and/or at least one pair plot comparing key statistics related to the ranking procedure.

The processing unit 112 is configured for determining the analysis model by training the machine learning model with the training data set. The training may comprise at least one optimization or tuning process, wherein a best parameter combination is determined. The training may be performed iteratively on the training data sets of different subjects. The processing unit 112 may be configured for considering different numbers of features for determining the analysis model by training the machine learning model with the training data set. The algorithm of the machine learning model may be applied to the training data set using a different number of features, e.g. depending on their ranking. The training may comprise n-fold cross validation to get a robust estimate of the model parameters. The training of the machine learning model may comprise at least one controlled learning process, wherein at least one hyper-parameter is chosen to control the training process. If necessary the training is step is repeated to test different combinations of hyper-parameters.

In particular subsequent to the training of the machine learning model, the processing unit 112 is configured for predicting the target variable on the test data set using the determined analysis model. The processing unit 112 may be configured for predicting the target variable for each subject based on the test data set of that subject using the determined analysis model. The processing unit 112 may be configured for predicting the target variable for each subject on the respective training and test data sets using the analysis model. The processing unit 112 may be configured for recording and/or storing both the predicted target variable per subject and the true value of the target variable per subject, for example, in at least one output file.

The processing unit 112 is configured for determining performance of the determined analysis model based on the predicted target variable and the true value of the target variable of the test data set. The performance may be characterized by deviations between predicted target variable and true value of the target variable. The machine learning system 110 may comprises at least one output interface 118. The output interface 118 may be designed identical to the communication interface 114 and/or may be formed integral with the communication interface 114. The output interface 118 may be configured for providing at least one output. The output may comprise at least one information about the performance of the determined analysis model. The information about the performance of the determined analysis model may comprises one or more of at least one scoring chart, at least one predictions plot, at least one correlations plot, and at least one residuals plot.

The model unit 116 may comprise a plurality of machine learning models, wherein the machine learning models are distinguished by their algorithm. For example, for building a regression model the model unit 116 may comprise the following algorithms k nearest neighbors (kNN), linear regression, partial last-squares (PLS), random forest (RF), and extremely randomized Trees (XT). For example, for building a classification model the model unit 116 may comprise the following algorithms k nearest neighbors (kNN), support vector machines (SVM), linear discriminant analysis (LDA), quadratic discriminant analysis (QDA), naïve Bayes (NB), random forest (RF), and extremely randomized Trees (XT). The processing unit 112 may be configured for determining a analysis model for each of the machine learning models by training the respective machine learning model with the training data set and for predicting the target variables on the test data set using the determined analysis models.

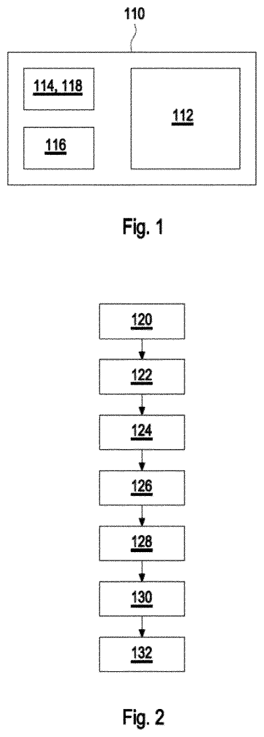

FIG. 2 shows an exemplary sequence of steps of a method according to the present invention. In step a), denoted with reference number 120, the input data is received via the communication interface 114. The method comprises pre-processing the input data, denoted with reference number 122. As outlined above, the pre-processing may comprise at least one filtering process for input data fulfilling at least one quality criterion. For example, the input data may be filtered to remove missing variables. For example, the pre-processing may comprise excluding data from subjects with less than a pre-defined minimum number of observations. In step b), denoted with reference number 124, the training data set and the test data set are determined by the processing unit 112. The method may further comprise at least one data aggregation and/or data transformation on both of the training data set and the test data set for each subject. The method may further comprise at least one feature extraction. The steps of data aggregation and/or data transformation and feature extraction are denoted with reference number 126 in FIG. 2. The feature extraction may comprise the ranking of features. In step c), denoted with reference number 128, the analysis model is determined by training a machine learning model comprising at least one algorithm with the training data set. In step d), denoted with reference number 130, the target variable is predicted on the test data set using the determined analysis model. In step e), denoted with reference number 132, performance of the determined analysis model is determined based on the predicted target variable and a true value of the target variable of the test data set

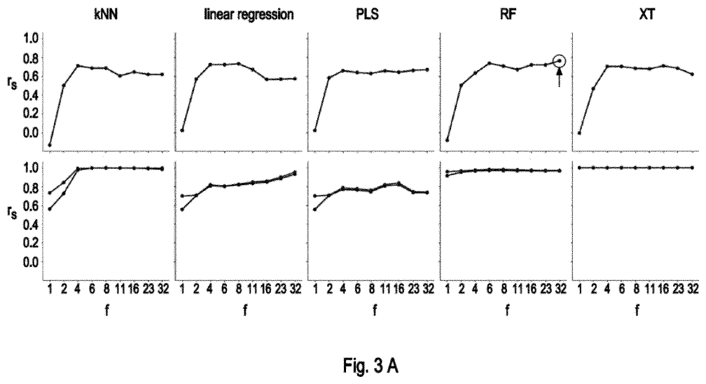

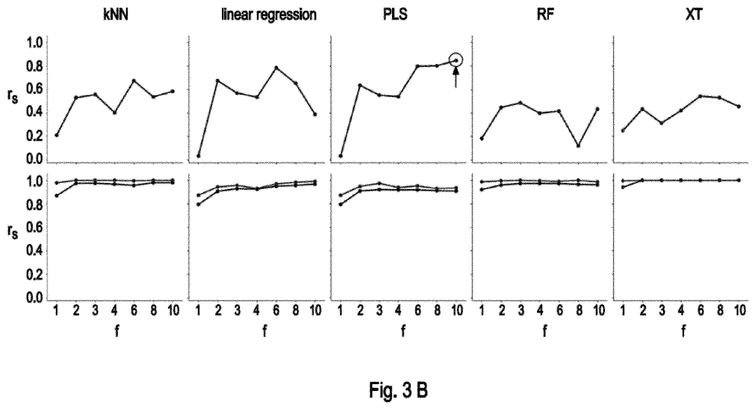

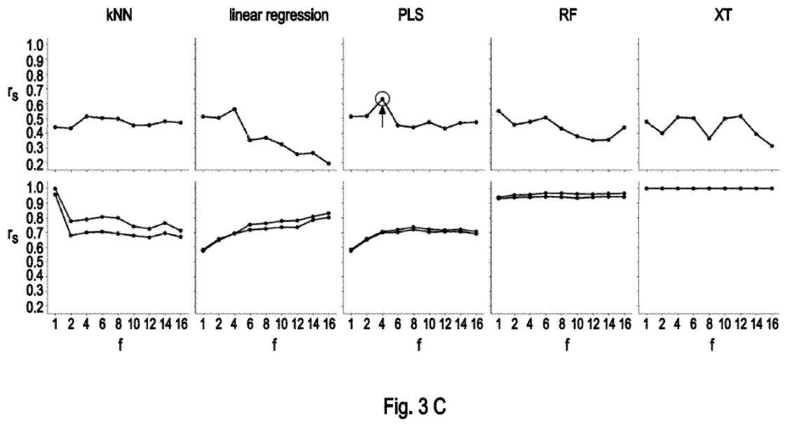

FIGS. 3A to 3C show embodiments of correlations plots for assessment of performance of an analysis model.

FIG. 3A show a correlations plot for analysis models, in particular regression models, for predicting an expanded disability status scale value indicative of multiple sclerosis. The input data was data from Floodlight POC study from 52 subjects.

In the prospective pilot study (FLOODLIGHT) the feasibility of conducting remote patient monitoring with the use of digital technology in patients with multiple sclerosis was evaluated. A study population was selected by using the following inclusion and exclusion criteria:
Key inclusion criteria:
Signed informed consent form
Able to comply with the study protocol, in the investigator's judgment
Age 18-55 years, inclusive
Have a definite diagnosis of MS, confirmed as per the revised McDonald 2010 criteria EDSS score of 0.0 to 5.5, inclusive
Weight: 45-110 kg
For women of childbearing potential: Agreement to use an acceptable birth control method during the study period
Key exclusion criteria:
Severely ill and unstable patients as per investigator's discretion
Change in dosing regimen or switch of disease modifying therapy (DMT) in the last 12 weeks prior to enrollment
Pregnant or lactating, or intending to become pregnant during the study

It is a primary objective of this study to show adherence to smartphone and smartwatch-based assessments quantified as compliance level (%) and to obtain feedback from patients and healthy controls on the smartphone and smartwatch schedule of assessments and the impact on their daily activities using a satisfaction questionnaire. Furthermore, additional objectives are addressed, in particular, the association between assessments conducted using the Floodlight Test and conventional MS clinical outcomes was determined, it was established if Floodlight measures can be used as a marker for disease activity/progression and are associated with changes in MRI and clinical outcomes over time and it was determined if the Floodlight Test Battery can differentiate between patients with and without MS, and between phenotypes in patients with MS.

In addition to the active tests and passive monitoring, the following assessments were performed at each scheduled clinic visit:
Oral Version of SDMT
Fatigue Scale for Motor and Cognitive Functions (FSMC)
Timed 25-Foot Walk Test (T25-FW)
Berg Balance Scale (BBS)
9-Hole Peg Test (9HPT)
Patient Health Questionnaire (PHQ-9)
Patients with MS only:
Brain MRI (MSmetrix)
Expanded Disability Status Scale (EDSS)
Patient Determined Disease Steps (PDDS)
Pen and paper version of MSIS-29

While performing in-clinic tests, patients and healthy controls were asked to carry/wear smartphone and smartwatch to collect sensor data along with in-clinic measures. In summary, the results of the study showed that patients are highly engaged with the smartphone- and smartwatch-based assessments. Moreover, there is a correlation between tests and in-clinic clinical outcome measures recorded at baseline which suggests that the smartphone-based Floodlight Test Battery shall become a powerful tool to continuously monitor MS in a real-world scenario. Further, the smartphone-based measurement of turning speed while walking and performing U-turns appeared to correlate with EDSS.

For FIG. 3A, in total, 889 features from 7 tests were evaluated during model building using the method according to the present invention. The tests used for this prediction were the Symbol-Digits Modalities Test (SM DT) where the subject has to match as many symbols as possible to digits in a given time span; the pinching test, where the subject has to squeeze, using the thumb and index finger, as many tomatoes shown on the screen as possible in a given time span; the Draw-A-Shape test, where the subject has to trace shapes on the screen; the Standing Balance Test where the subject has to stand upright for 30 seconds; the 5 U-Turn test where the subject has to walk short spans followed by 180 degree turns; the 2 Minute Walking test, where the subject has to walk for two minutes; and finally the passive monitoring of the gait. The following table gives an overview of selected features used for prediction, test from which the feature was derived, short description of feature and ranking:

| feature | test | Description of feature | rank |
|---|---|---|---|
| logistic | Passive | Average per-step power coefficient | 1 |
| step_power_mean | Monitoring | (integral of variance in accelerometer | |
| (40-60 s) | | radius over per-step time span) for | |
| | | gait bouts spanning 40-60 s | |
| sigmoid turns_utt | U-TURN | Number of turns | 2 |
| log10 Gc_0_15 | SDMT | Mean Timegap between correct | 3 |
| | | responses from time 0 to 15 seconds | |
| sigmoid | U-TURN | maximum turn speed | 4 |
| turn_speed_max_utt | | | |
| logistic | 2MWT | Average per-step power coefficient | 5 |
| step_power_mean | | (integral of variance in accelerometer | |
| | | radius over per-step time span) | |
| sigmoid | U-TURN | minimum turn speed | 6 |
| turn_speed_min_utt | | | |
| sigmoid | Passive | Variance of per-step power coefficient | 7 |
| step_power_variance | Monitoring | for gait bouts spanning 60-90 s | |
| (60-90 s) | | | |
| logistic | Passive | Variance of per-step power coefficient | 8 |
| step_power_variance | Monitoring | for gait bouts spanning 40-60 s | |
| (40-60 s) | | | |
| sigmoid | Passive | Average per-step power coefficient | 9 |
| step_power_mean | Monitoring | (integral of variance in accelerometer | |
| (<20 s) | | radius over per-step time span) for | |
| | | gait bouts spanning <20 s | |
| span_dura- | U-TURN | median gait bout length | 10 |
| tion_s_median_utt | | | |
| logistic | Passive | Variance of per-step power coefficient | 11 |
| step_power_variance | Monitoring | for gait bouts spanning 20-40 s | |
| (20-40 s) | | | |
| sigmoid | Passive | Variance of per-step power coefficient | 12 |
| step_power_variance | Monitoring | for gait bouts spanning 90-120 s | |
| (90-120 s) | | | |
| sigmoid | U-TURN | median turn speed | 13 |
| turn_speed_median_utt | | | |
| logistic | Passive | Average per-step power coefficient | 14 |
| step_power_mean | Monitoring | (integral of variance in accelerometer | |
| (60-90 s) | | radius over per-step time span) for | |
| | | gait bouts spanning 60-90 s | |
| sigmoid GcM_0_15 | SDMT | Maximal Timegap between correct | 15 |
| | | responses from time 0 to 15 seconds | |
| logistic | Passive | Average per-step power coefficient | 16 |
| step_power_mean | Monitoring | (integral of variance in accelerometer | |
| (20-40 s) | | radius over per-step time span) for | |
| | | gait bouts spanning 20-40 s | |
| logistic | Passive | Average per-step power coefficient | 17 |
| step_power_mean | Monitoring | (integral of variance in accelerometer | |
| (90-120 s) | | radius over per-step time span) for | |
| | | gait bouts spanning 90-120 s | |
| CCR_0_45 | SDMT | from time 0 to 45 seconds: Number of | 18 |
| | | correct responses within the longest | |
| | | sequence of overall consecutive correct | |
| | | responses | |
| span_dura- | U-TURN | maximum gait bout length | 19 |
| tion_s_max_utt | | | |
| log10 R_Symbol_9 | SDMT | Number of total responses for symbol | 20 |
| | | 9: “.-” | |
| Gc_0_30 | SDMT | Mean Timegap between correct | 21 |
| | | responses from time 0 to 30 seconds | |
| sigmoid CCR_0_15 | SDMT | from time 0 to 15 seconds: Number of | 22 |
| | | correct responses within the longest | |
| | | sequence of overall consecutive correct | |
| | | responses | |
| sigmoid GM_0_15 | SDMT | Maximal Timegap between responses | 23 |
| | | from time 0 to 15 seconds | |
| sigmoid R_0_15 | SDMT | Number of total responses from time | 24 |
| | | 0 to 15 seconds | |
| log10 CR_Symbol_8 | SDMT | Number of correct responses for symbol | 25 |
| | | 8: “)” | |
| log10 CCR_0_30 | SDMT | from time 0 to 30 seconds: Number of | 26 |
| | | correct responses within the longest | |
| | | sequence of overall consecutive correct | |
| | | responses | |
| log10 G_0_15 | SDMT | Mean Timegap between responses | 27 |
| | | from time 0 to 15 seconds | |
| sigmoid CR_0_15 | SDMT | Number of correct responses from | 28 |
| | | time 0 to 15 seconds | |
| log10 Gc_0_45 | SDMT | Mean Timegap between correct | 29 |
| | | responses from time 0 to 45 seconds | |
| log10 R_Symbol_8 | SDMT | Number of total responses for symbol | 30 |
| | | 8: “)” | |
| log10 R_0_30 | SDMT | Number of total responses from time | 31 |
| | | 0 to 30 seconds | |
| sigmoid CR_0_30 | SDMT | Number of correct responses from | 32 |
| | | time 0 to 30 seconds | |

FIG. 3A shows the Spearman correlation coefficient rₛ between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is RF with 32 features included in the model, having an rₛ value of 0.77, indicated with circle and arrow.

The following gives more detailed description of the tests. The tests are typically computer-implemented on a data acquisition device such as a mobile device as specified elsewhere herein.

(1) Tests for Passive Monitoring of Gait and Posture: Passive Monitoring

The mobile device is, typically, adapted for performing or acquiring data from passive monitoring of all or a subset of activities In particular, the passive monitoring shall encompass monitoring one or more activities performed during a predefined window, such as one or more days or one or more weeks, selected from the group consisting of: measurements of gait, the amount of movement in daily routines in general, the types of movement in daily routines, general mobility in daily living and changes in moving behavior.

Typical passive monitoring performance parameters of interest:
a. frequency and/or velocity of walking;
b. amount, ability and/or velocity to stand up/sit down, stand still and balance
c. number of visited locations as an indicator of general mobility;
d. types of locations visited as an indicator of moving behavior.

(2) Test for Cognitive Capabilities: SMDT (Also Denoted as eSDMT)

The mobile device is also, typically, adapted for performing or acquiring a data from an computer-implemented Symbol Digit Modalities Test (eSDMT). The conventional paper SDMT version of the test consists of a sequence of 120 symbols to be displayed in a maximum 90 seconds and a reference key legend (3 versions are available) with 9 symbols in a given order and their respective matching digits from 1 to 9. The smartphone-based eSDMT is meant to be self-administered by patients and will use a sequence of symbols, typically, the same sequence of 110 symbols, and a random alternation (form one test to the next) between reference key legends, typically, the 3 reference key legends, of the paper/oral version of SDMT. The eSDMT similarly to the paper/oral version measures the speed (number of correct paired responses) to pair abstract symbols with specific digits in a predetermined time window, such as 90 seconds time. The test is, typically, performed weekly but could alternatively be performed at higher (e.g. daily) or lower (e.g. bi-weekly) frequency. The test could also alternatively encompass more than 110 symbols and more and/or evolutionary versions of reference key legends. The symbol sequence could also be administered randomly or according to any other modified pre-specified sequence.

Typical eSDMT performance parameters of interest:
1. Number of correct responses
   a. Total number of overall correct responses (CR) in 90 seconds (similar to oral/paper SDMT)
   b. Number of correct responses from time 0 to 30 seconds (CR₀₋₃₀)
   c. Number of correct responses from time 30 to 60 seconds (CR₃₀₋₆₀)
   d. Number of correct responses from time 60 to 90 seconds (CR₆₀₋₉₀)
   e. Number of correct responses from time 0 to 45 seconds (CR₀₋₄₅)
   f. Number of correct responses from time 45 to 90 seconds (CR₄₅₋₉₀)
   g. Number of correct responses from time i to j seconds (CRᵢ₋ⱼ), where i,j are between 1 and 90 seconds and i<j.
2. Number of errors
   a. Total number of errors (E) in 90 seconds
   b. Number of errors from time 0 to 30 seconds (E₀₋₃₀)
   c. Number of errors from time 30 to 60 seconds (E₃₀₋₆₀)
   d. Number of errors from time 60 to 90 seconds (E₆₀₋₉₀)
   e. Number of errors from time 0 to 45 seconds (E₀₋₄₅)
   f. Number of errors from time 45 to 90 seconds (E₄₅₋₉₀)
   g. Number of errors from time i to j seconds (Eᵢ₋ⱼ), where i,j are between 1 and 90 seconds and i<j.
3. Number of responses
   a. Total number of overall responses (R) in 90 seconds
   b. Number of responses from time 0 to 30 seconds (R₀₋₃₀)
   c. Number of responses from time 30 to 60 seconds (R₃₀₋₆₀)
   d. Number of responses from time 60 to 90 seconds (R₆₀₋₉₀)
   e. Number of responses from time 0 to 45 seconds (R₀₋₄₅)
   f. Number of responses from time 45 to 90 seconds (R₄₅₋₉₀)
4. Accuracy rate
   a. Mean accuracy rate (AR) over 90 seconds: AR=CR/R
   b. Mean accuracy rate (AR) from time 0 to 30 seconds: AR₀₋₃₀=CR₀₋₃₀/R₀₋₃₀
   c. Mean accuracy rate (AR) from time 30 to 60 seconds: AR₃₀₋₆₀=CR₃₀₋₆₀/R₃₀₋₆₀
   d. Mean accuracy rate (AR) from time 60 to 90 seconds: AR₆₀₋₉₀=CR₆₀₋₉₀/R₆₀₋₉₀
   e. Mean accuracy rate (AR) from time 0 to 45 seconds: AR₀₋₄₅=CR₀₋₄₅/R₀₋₄₅
   f. Mean accuracy rate (AR) from time 45 to 90 seconds: AR₄₅₋₉₀=CR₄₅₋₉₀/R₄₅₋₉₀
5. End of task fatigability indices
   a. Speed Fatigability Index (SFI) in last 30 seconds: SFI₆₀₋₉₀=CR₆₀₋₉₀/max (CR₀₋₃₀, CR₃₀₋₆₀)
   b. SFI in last 45 seconds: SFI₄₅₋₉₀=CR₄₅₋₉₀/CR₀₋₄₅
   c. Accuracy Fatigability Index (AFI) in last 30 seconds: AFI₆₀₋₉₀=AR₆₀₋₉₀/max (AR₀₋₃₀, AR₃₀₋₆₀)
   d. AFI in last 45 seconds: AFI₄₅₋₉₀=AR₄₅₋₉₀/AR₀₋₄₅
6. Longest sequence of consecutive correct responses
   a. Number of correct responses within the longest sequence of overall consecutive correct responses (CCR) in 90 seconds
   b. Number of correct responses within the longest sequence of consecutive correct responses from time 0 to 30 seconds (CCR₀₋₃₀)
   c. Number of correct responses within the longest sequence of consecutive correct responses from time 30 to 60 seconds (CCR₃₀₋₆₀)
   d. Number of correct responses within the longest sequence of consecutive correct responses from time 60 to 90 seconds (CCR₆₀₋₉₀)
   e. Number of correct responses within the longest sequence of consecutive correct responses from time 0 to 45 seconds (CCR₀₋₄₅)
   f. Number of correct responses within the longest sequence of consecutive correct responses from time 45 to 90 seconds (CCR₄₅₋₉₀)
7. Time gap between responses
   a. Continuous variable analysis of gap (G) time between two successive responses
   b. Maximal gap (GM) time elapsed between two successive responses over 90 seconds
   c. Maximal gap time elapsed between two successive responses from time 0 to seconds (GM₀₋₃₀)
   d. Maximal gap time elapsed between two successive responses from time 30 to 60 seconds (GM₃₀₋₆₀)
   e. Maximal gap time elapsed between two successive responses from time 60 to 90 seconds (GM₆₀₋₉₀)
   f. Maximal gap time elapsed between two successive responses from time 0 to seconds (GM₀₋₄₅)
   g. Maximal gap time elapsed between two successive responses from time 45 to 90 seconds (GM₄₅₋₉₀)
8. Time Gap between correct responses
   a. Continuous variable analysis of gap (Gc) time between two successive correct responses
   b. Maximal gap time elapsed between two successive correct responses (GcM) over 90 seconds
   c. Maximal gap time elapsed between two successive correct responses from time 0 to 30 seconds (GcM₀₋₃₀)
   d. Maximal gap time elapsed between two successive correct responses from time 30 to 60 seconds (GcM₃₀₋₆₀)
   e. Maximal gap time elapsed between two successive correct responses from time 60 to 90 seconds (GcM₆₀₋₉₀)
   f. Maximal gap time elapsed between two successive correct responses from time 0 to 45 seconds (GcM₀₋₄₅)
      g. Maximal gap time elapsed between two successive correct responses from time 45 to 90 seconds (GcM₄₅₋₉₀)
9. Fine finger motor skill function parameters captured during eSDMT
   a. Continuous variable analysis of duration of touchscreen contacts (Tts), deviation between touchscreen contacts (Dts) and center of closest target digit key, and mistyped touchscreen contacts (Mts) (i.econtacts not triggering key hit or triggering key hit but associated with secondary sliding on screen), while typing responses over 90 seconds
   b. Respective variables by epochs from time 0 to 30 seconds: Tts₀₋₃₀, Dts₀₋₃₀, Mts₀₋₃₀
   c. Respective variables by epochs from time 30 to 60 seconds: Tts₃₀₋₆₀, Dts₃₀₋₆₀, Mts₃₀₋₆₀
   d. Respective variables by epochs from time 60 to 90 seconds: Tts₆₀₋₉₀, Dts₆₀₋₉₀, Mts₆₀₋₉₀
   e. Respective variables by epochs from time 0 to 45 seconds: Tts₀₋₄₅, Dts₀₋₄₅, Mts₀₋₄₅
   f. Respective variables by epochs from time 45 to 90 seconds: Tts₄₅₋₉₀, Dts₄₅₋₉₀, MtS₄₅₋₉₀
10. Symbol-specific analysis of performances by single symbol or cluster of symbols
   a. CR for each of the 9 symbols individually and all their possible clustered combinations
   b. AR for each of the 9 symbols individually and all their possible clustered combinations
   c. Gap time (G) from prior response to recorded responses for each of the 9 symbols individually and all their possible clustered combinations
   d. Pattern analysis to recognize preferential incorrect responses by exploring the type of mistaken substitutions for the 9 symbols individually and the 9 digit responses individually.
11. Learning and cognitive reserve analysis
   a. Change from baseline (baseline defined as the mean performance from the first 2 administrations of the test) in CR (overall and symbol-specific as described in #9) between successive administrations of eSDMT
   b. Change from baseline (baseline defined as the mean performance from the first 2 administrations of the test) in AR (overall and symbol-specific as described in #9) between successive administrations of eSDMT
   c. Change from baseline (baseline defined as the mean performance from the first 2 administrations of the test) in mean G and GM (overall and symbol-specific as described in #9) between successive administrations of eSDMT
   d. Change from baseline (baseline defined as the mean performance from the first 2 administrations of the test) in mean Gc and GcM (overall and symbol-specific as described in #9) between successive administrations of eSDMT
   e. Change from baseline (baseline defined as the mean performance from the first 2 administrations of the test) in SFI₆₀₋₉₀ and SFI₄₅₋₉₀ between successive administrations of eSDMT
   f. Change from baseline (baseline defined as the mean performance from the first 2 administrations of the test) in AFI₆₀₋₉₀ and AFI₄₅₋₉₀ between successive administrations of eSDMT
   g. Change from baseline (baseline defined as the mean performance from the first 2 administrations of the test) in Tts between successive administrations of eSDMT
   h. Change from baseline (baseline defined as the mean performance from the first 2 administrations of the test) in Dts between successive administrations of eSDMT
   i. Change from baseline (baseline defined as the mean performance from the first 2 administrations of the test) in Mts between successive administrations of eSDMT.
(3) Tests for active gait and posture capabilities: U-Turn Test (also denoted as Five U-Turn Test, 5UTT) and 2MWT

A sensor-based (e.g. accelerometer, gyroscope, magnetometer, global positioning system [GPS]) and computer implemented test for measures of ambulation performances and gait and stride dynamics, in particular, the 2-Minute Walking Test (2MWT) and the Five U-Turn Test (5UTT).

In one embodiment, the mobile device is adapted to perform or acquire data from the Two-Minute Walking Test (2MWT). The aim of this test is to assess difficulties, fatigability or unusual patterns in long-distance walking by capturing gait features in a two-minute walk test (2MWT). Data will be captured from the mobile device. A decrease of stride and step length, increase in stride duration, increase in step duration and asymmetry and less periodic strides and steps may be observed in case of disability progression or emerging relapse. Arm swing dynamic while walking will also be assessed via the mobile device. The subject will be instructed to “walk as fast and as long as you can for 2 minutes but walk safely”. The 2MWT is a simple test that is required to be performed indoor or outdoor, on an even ground in a place where patients have identified they could walk straight for as far as ≥200 meters without U-turns. Subjects are allowed to wear regular footwear and an assistive device and/or orthotic as needed. The test is typically performed daily.

Typical 2MWT performance parameters of particular interest:
1. Surrogate of walking speed and spasticity:
   a. Total number of steps detected in, e.g., 2 minutes (ES)
   b. Total number of rest stops if any detected in 2 minutes (ERs)
   c. Continuous variable analysis of walking step time (WsT) duration throughout the 2MWT
   d. Continuous variable analysis of walking step velocity (WsV) throughout the 2MWT (step/second)
   e. Step asymmetry rate throughout the 2MWT (mean difference of step duration between one step to the next divided by mean step duration): SAR=meanΔ(WsTₓ−WsTₓ₊₁)/(120/ΣS)
   f. Total number of steps detected for each epoch of 20 seconds (ΣS_{t,t+20})
   g. Mean walking step time duration in each epoch of 20 seconds: WsT_{t,t+20}=20/ΣS_{t, t+20}
   h. Mean walking step velocity in each epoch of 20 seconds: WsV_{t,t+20}=ΣS_{t,t+20}/20
   i. Step asymmetry rate in each epoch of 20 seconds: SAR_{t, t+20}=meanΔ_{t,t+20}(WsTₓ−WsTₓ₊₁)/(20/ΣS_{t, t+20})
   j. Step length and total distance walked through biomechanical modelling
2. Walking fatigability indices:
   a. Deceleration index: DI=WsV₁₀₀₋₁₂₀/max (WsV₀₋₂₀, WsV₂₀₋₄₀, WsV₄₀₋₆₀
   b. Asymmetry index: AI=SAR₁₀₀₋₁₂₀/min (SAR₀₋₂₀, SAR₂₀₋₄₀, SAR₄₀₋₆₀)

In another embodiment, the mobile device is adapted to perform or acquire data from the Five U-Turn Test (5UTT). The aim of this test is to assess difficulties or unusual patterns in performing U-turns while walking on a short distance at comfortable pace. The 5UTT is required to be performed indoor or outdoor, on an even ground where patients are instructed to “walk safely and perform five successive U-turns going back and forward between two points a few meters apart”. Gait feature data (change in step counts, step duration and asymmetry during U-turns, U-turn duration, turning speed and change in arm swing during U-turns) during this task will be captured by the mobile device. Subjects are allowed to wear regular footwear and an assistive device and/or orthotic as needed. The test is typically performed daily.

Typical 5UTT performance parameters of interest:
1. Mean number of steps needed from start to end of complete U-turn (ΣSu)
2. Mean time needed from start to end of complete U-turn (Tu)
3. Mean walking step duration: Tsu=Tu/ΣSu
4. Turn direction (left/right)
5. Turning speed (degrees/sec)

FIG. 3B show a correlations plot for analysis models, in particular regression models, for predicting a forced vital capacity (FVC) value indicative of spinal muscular atrophy. The input data was data from OLEOS study from 14 subjects. In total, 1326 features from 9 tests were evaluated during model building using the method according to the present invention. The following table gives an overview of selected features used for prediction, test from which the feature was derived, short description of feature and ranking:

| Performance parameter | test | description | rank |
|---|---|---|---|
| Imax_pressure_min | Distal Motor | The minimum value of | 1 |
| | Function test | each maximum pressure | |
| | (Tap-The- | reading per finger tap | |
| | Monster) | | |
| log10 DTA_F | Squeeze-A- | the mean lag time between | 2 |
| | Shape | first and second fingers | |
| | | touch the screen of failed | |
| | | pinches | |
| log10 | Voice test | Mean absolute difference | 3 |
| norm_pct_diff_Mean_MFCCs_9 | | of successive cycles of the | |
| | | 9^{th} Mel Frequency Cepstral | |
| | | Coefficient (MFCC) | |
| log10 std_Mean_MFCCs_8 | Voice test | The standard deviation of | 4 |
| | | the mean value of successive | |
| | | cycles of the 8th MFCC | |
| logistic fatigue_index | Voice test | An estimate for vocal | 5 |
| | | fatigue defined as the ratio | |
| | | of max duration of the first | |
| | | half to max duration of the | |
| | | second half | |
| log10 DTA_S | Squeeze-A- | the mean lag time between | 6 |
| | Shape | first and second fingers | |
| | | touch the screen of | |
| | | successful pinches | |
| sigmoid | Draw-A- | square root of the drawing | 7 |
| LINE_TOP_TO_BOT- | Shape | error for the line top-to- | |
| TOM_errSQRT | | bottom shape | |
| log10 DTA_0_15 | Squeeze-A- | the mean lag time between | 8 |
| | Shape | first and second fingers | |
| | | touch the screen between | |
| | | time window 0 s-15 s | |
| log10 DTA_15_30 | Squeeze-A- | the mean lag time between | 9 |
| | Shape | first and second fingers | |
| | | touch the screen between | |
| | | time window 15 s-30 s | |
| log10 DTA | Squeeze-A- | DTA = mean(pinch_start − | 10 |
| | Shape | finger_down): the mean | |
| | | lag time between first and | |
| | | second fingers touch the | |
| | | screen | |

FIG. 3B shows the Spearman correlation coefficient r s between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is PLS with 10 features included in the model, having an rₛ value of 0.8, indicated with circle and arrow.

The following gives more detailed description of the tests. The tests are typically computer-implemented on a data acquisition device such as a mobile device as specified elsewhere herein.

(1) Tests for Central Motor Functions: Draw a Shape Test and Squeeze a Shape Test

The mobile device may be further adapted for performing or acquiring a data from a further test for distal motor function (so-called “draw a shape test”) configured to measure dexterity and distal weakness of the fingers. The dataset acquired from such test allow identifying the precision of finger movements, pressure profile and speed profile.

The aim of the “Draw a Shape” test is to assess fine finger control and stroke sequencing. The test is considered to cover the following aspects of impaired hand motor function: tremor and spasticity and impaired hand-eye coordination. The patients are instructed to hold the mobile device in the untested hand and draw on a touchscreen of the mobile device 6 pre-written alternating shapes of increasing complexity (linear, rectangular, circular, sinusoidal, and spiral; vide infra) with the second finger of the tested hand “as fast and as accurately as possible” within a maximum time of for instance 30 seconds. To draw a shape successfully the patient's finger has to slide continuously on the touchscreen and connect indicated start and end points passing through all indicated check points and keeping within the boundaries of the writing path as much as possible. The patient has maximum two attempts to successfully complete each of the 6 shapes. Test will be alternatingly performed with right and left hand. User will be instructed on daily alternation. The two linear shapes have each a specific number “a” of checkpoints to connect, i.e “a-1” segments. The square shape has a specific number “b” of checkpoints to connect, i.e. “b-1” segments. The circular shape has a specific number “c” of checkpoints to connect, i.e. “c-1” segments. The eight-shape has a specific number “d” of checkpoints to connect, i.e “d-1” segments. The spiral shape has a specific number “e” of checkpoints to connect, “e-1” segments. Completing the 6 shapes then implies to draw successfully a total of “(2a+b+c+d+e-6)” segments.

Typical Draw a Shape test performance parameters of interest:

Based on shape complexity, the linear and square shapes can be associated with a weighting factor (Wf) of 1, circular and sinusoidal shapes a weighting factor of 2, and the spiral shape a weighting factor of 3. A shape which is successfully completed on the second attempt can be associated with a weighting factor of 0.5. These weighting factors are numerical examples which can be changed in the context of the present invention.
1. Shape completion performance scores:
   a. Number of successfully completed shapes (0 to 6) (ΣSh) per test
   b. Number of shapes successfully completed at first attempt (0 to 6) (ΣSh₁)
   c. Number of shapes successfully completed at second attempt (0 to 6) (ΣSh₂)
   d. Number of failed/uncompleted shapes on all attempts (0 to 12) (ΣF)
   e. Shape completion score reflecting the number of successfully completed shapes adjusted with weighting factors for different complexity levels for respective shapes (0 to 10) (Σ[Sh*Wf])
   f. Shape completion score reflecting the number of successfully completed shapes adjusted with weighting factors for different complexity levels for respective shapes and accounting for success at first vs second attempts (0 to 10) (Σ[Sh₁*Wf]+Σ[Sh₂*Wf*0.5])
   g. Shape completion scores as defined in #1e, and #1f may account for speed at test completion if being multiplied by 30/t, where t would represent the time in seconds to complete the test.
   h. Overall and first attempt completion rate for each 6 individual shapes based on multiple testing within a certain period of time: (ΣSh₁)/(ΣSh₁+ΣSh₂+ΣF) and (ΣSh₁+ΣSh₂)/(ΣSh₁+ΣSh₂+ΣF).
2. Segment completion and celerity performance scores/measures:
(analysis based on best of two attempts [highest number of completed segments] for each shape, if applicable)
   a. Number of successfully completed segments (0 to [2a+b+c+d+e-6]) (ΣSe) per test
   b. Mean celerity ([C], segments/second) of successfully completed segments: C =ΣSe/t, where t would represent the time in seconds to complete the test (max 30 seconds)
   c. Segment completion score reflecting the number of successfully completed segments adjusted with weighting factors for different complexity levels for respective shapes (Σ[Se*Wf])
   d. Speed-adjusted and weighted segment completion score (Σ[Se*Wf]*30/t), where t would represent the time in seconds to complete the test.
   e. Shape-specific number of successfully completed segments for linear and square shapes (ΣSe_{LS})
   f. Shape-specific number of successfully completed segments for circular and sinusoidal shapes (ΣSe_{CS})
   g. Shape-specific number of successfully completed segments for spiral shape (ΣSe_{S})
   h. Shape-specific mean linear celerity for successfully completed segments performed in linear and square shape testing: C_{L}=ΣSe_{LS}/t, where t would represent the cumulative epoch time in seconds elapsed from starting to finishing points of the corresponding successfully completed segments within these specific shapes.
   i. Shape-specific mean circular celerity for successfully completed segments performed in circular and sinusoidal shape testing: C_{C}=ΣSe_{CS}/t, where t would represent the cumulative epoch time in seconds elapsed from starting to finishing points of the corresponding successfully completed segments within these specific shapes.
   j. Shape-specific mean spiral celerity for successfully completed segments performed in the spiral shape testing: C_{S}=ESe_{S}/t, where t would represent the cumulative epoch time in seconds elapsed from starting to finishing points of the corresponding successfully completed segments within this specific shape.
3. Drawing precision performance scores/measures: (analysis based on best of two attempts[highest number of completed segments] for each shape, if applicable)
   a. Deviation (Dev) calculated as the sum of overall area under the curve (AUC) measures of integrated surface deviations between the drawn trajectory and the target drawing path from starting to ending checkpoints that were reached for each specific shapes divided by the total cumulative length of the corresponding target path within these shapes (from starting to ending checkpoints that were reached).
   b. Linear deviation (Dev_{L}) calculated as Dev in #3a but specifically from the linear and square shape testing results.
   c. Circular deviation (Dev_{C}) calculated as Dev in #3a but specifically from the circular and sinusoidal shape testing results.
   d. Spiral deviation (Dev_{S}) calculated as Dev in #3a but specifically from the spiral shape testing results.
   e. Shape-specific deviation (Dev₁₋₆) calculated as Dev in #3a but from each of the 6 distinct shape testing results separately, only applicable for those shapes where at least 3 segments were successfully completed within the best attempt.
   f. Continuous variable analysis of any other methods of calculating shape-specific or shape-agnostic overall deviation from the target trajectory.
4.) Pressure profile measurement
i) Exerted average pressure
ii) Deviation (Dev) calculated as the standard deviation of pressure

The distal motor function (so-called “squeeze a shape test”) may measure dexterity and distal weakness of the fingers. The dataset acquired from such test allow identifying the precision and speed of finger movements and related pressure profiles. The test may require calibration with respect to the movement precision ability of the subject first.

The aim of the Squeeze a Shape test is to assess fine distal motor manipulation (gripping & grasping) & control by evaluating accuracy of pinch closed finger movement. The test is considered to cover the following aspects of impaired hand motor function: impaired gripping/grasping function, muscle weakness, and impaired hand-eye coordination. The patients are instructed to hold the mobile device in the untested hand and by touching the screen with two fingers from the same hand (thumb+second or thumb+third finger preferred) to squeeze/pinch as many round shapes (i.e. tomatoes) as they can during 30 seconds. Impaired fine motor manipulation will affect the performance. Test will be alternatingly performed with right and left hand. User will be instructed on daily alternation.

Typical Squeeze a Shape test performance parameters of interest:
1. Number of squeezed shapes
   a. Total number of tomato shapes squeezed in 30 seconds (ΣSh)
   b. Total number of tomatoes squeezed at first attempt (ΣSh₁) in 30 seconds (a first attempt is detected as the first double contact on screen following a successful squeezing if not the very first attempt of the test)
2. Pinching precision measures:
   a. Pinching success rate (P_{SR}) defined as ΣSh divided by the total number of pinching (ΣP) attempts (measured as the total number of separately detected double finger contacts on screen) within the total duration of the test.
   b. Double touching asynchrony (DTA) measured as the lag time between first and second fingers touch the screen for all double contacts detected.
   c. Pinching target precision (P_{TP}) measured as the distance from equidistant point between the starting touch points of the two fingers at double contact to the centre of the tomato shape, for all double contacts detected.
   d. Pinching finger movement asymmetry (P_{FMA}) measured as the ratio between respective distances slid by the two fingers (shortest/longest) from the double contact starting points until reaching pinch gap, for all double contacts successfully pinching.
   e. Pinching finger velocity (P_{FV}) measured as the speed (mm/sec) of each one and/or both fingers sliding on the screen from time of double contact until reaching pinch gap, for all double contacts successfully pinching.
   f. Pinching finger asynchrony (P_{FA}) measured as the ratio between velocities of respective individual fingers sliding on the screen (slowest/fastest) from the time of double contact until reaching pinch gap, for all double contacts successfully pinching.
   g. Continuous variable analysis of 2a to 2f over time as well as their analysis by epochs of variable duration (5-15 seconds)
   h. Continuous variable analysis of integrated measures of deviation from target drawn trajectory for all tested shapes (in particular the spiral and square)
3.) Pressure profile measurement
i) Exerted average pressure
ii) Deviation (Dev) calculated as the standard deviation of pressure

More typically, the Squeeze a Shape test and the Draw a Shape test are performed in accordance with the method of the present invention. Even more specifically, the performance parameters listed in the Table 1 below are determined.

In addition to the features outlined above, various other features may also be evaluated when performing a “squeeze a shape” or “pinching” test. These are described below. The following terms are used in the description of the additional features:
Pinching Test: A digital upper limb/hand mobility test requiring pinching motions with the thumb and forefinger to squeeze a round shape on the screen.
Feature: A scalar value calculated from raw data collected by the smartphone during the single execution of a distal motor test. It is a digital measure of the subject's performance.
Stroke: Uninterrupted path drawn by a finger on the screen. The stroke starts when the finger touches the screen for the first time, and ends when the finger leaves the screen.
Gesture: Collection of all the Strokes registered between the first finger touching the screen, and the last finger leaving the screen.
Attempt: Any Gesture containing at least two Strokes. Such Gesture is considered to be an attempt to squeeze the round shape visible on the screen.
Two-Finger Attempt: Any Attempt with exactly two Strokes.
Successful Attempt: Any Attempt resulting in the round shape being registered as “squeezed”.

The features are as follows:
Distance between last points: for each attempt, the first two recorded strokes are kept, and for each pair, the distance between the last points in both strokes is calculated. This may be done for all attempts, or just the successful attempts.
End asymmetry: For each attempt, the first two recorded strokes are kept, and for each pair, the time difference between the first and the second finger leaving the screen is calculated.
Gap Times: For each pair of consecutive attempts, the duration of the gap between them is calculated. In other words, for each pair of attempts i and i+1, the time difference between the end of Attempt i and the beginning of Attempt i+1 is calculated.
Number of performed attempts: The number of performed attempts is returned.
Number of successful attempts: The number of successful attempts is returned.
Number of two-finger attempts: The number of two-finger attempts is returned. This may be divided by the total number of attempts, to return a two-finger attempts fraction.
Pinch times: For each attempt, the duration of the attempt is calculated. The duration is defined at the time between the first finger touching the screen and the last feature leaving the screen. This feature may also be defined as the duration for which both fingers are present on the screen.
Start asymmetry: For each attempt, the first two recorded strokes are kept. For each pair, the time difference between the first and second finger touching the screen is calculated.
Stroke Path Ratio: For each attempt, the first and second recorded strokes are kept. For each stroke, two values are calculated: the length of the path travelled by the finger on the screen, and the distance between the first and last point in the stroke. For each stroke, the ratio (path length/distance) is calculated. This may be done for all attempts, or just for successful attempts.

In all of the above cases, the test may be performed several times, and a statistical parameter such as the mean, standard deviation, kurtosis, median, and a percentile may be derived. Where a plurality of measurements are taken in this manner, a generic fatigue factor may be determined.
Generic fatigue feature: The data from the test is split into two halves of a predetermined duration each, e.g. 15 seconds. Any of the features defined above is calculated using the first and second half of the data separately, resulting in two feature values. The difference between the first and second value is returned. This may be normalized by dividing by the first feature value.

In some cases, the data acquisition device such as a mobile device may include an accelerometer, which may be configured to measure acceleration data during the period while the test is being performed. There are various useful features which can be extracted from the acceleration data too, as described below:
Horizontalness: For each time point, the z-component of the acceleration is divided by the total magnitude. The mean of the resulting time series may then be taken. The absolute value may be taken. Throughout this application, the z-component is defined as the component which is perpendicular to a plane of the touchscreen display.
Orientation stability: For each time point, the z-component of the acceleration is divided by the total magnitude. The standard deviation of the resulting time series may then be taken. The absolute value may be taken. Here, the z-component is defined as the component which is perpendicular to a plane of the touchscreen display.
Standard deviation of z-axis: For each time point, the z-component of the acceleration is measured. The standard deviation over the time series may then be taken.
Standard deviation of acceleration magnitude: For each time point, the x-, y-, and z-components of the acceleration are taken. The standard deviation over the x-component is taken. The standard deviation over the y-component is taken. The standard deviation over the z-component is taken. The norm of the standard deviations is then calculated by adding the three separate standard deviations in quadrature.
Acceleration magnitude: The total magnitude of the acceleration may be determined for the duration of the test. Then a statistical parameter may be derived either: over the whole duration of the test, or only for those time points when fingers are present on the screen, or only for those time points where no fingers are present on the screen. The statistical parameter may be the mean, standard deviation or kurtosis.

It should be stressed that, where possible, these acceleration-based features need not only be taken during a pinching or squeeze-a-shape, as they are able to yield clinically meaningful outputs independent of the kind of test during which they are extracted. This is especially true of the horizontalness and orientation stability parameters.

The data acquisition device may be further adapted for performing or acquiring a data from a further test for central motor function (so-called “voice test”) configured to measure proximal central motoric functions by measuring voicing capabilities.

(2) Cheer-The-Monster test, Voice test:

The term “Cheer-the-Monster test”, as used herein, relates to a test for sustained phonation, which is, in an embodiment, a surrogate test for respiratory function assessments to address abdominal and thoracic impairments, in an embodiment including voice pitch variation as an indicator of muscular fatigue, central hypotonia and/or ventilation problems. In an embodiment, Cheer-the-Monster measures the participant's ability to sustain a controlled vocalization of an “aaah” sound. The test uses an appropriate sensor to capture the participant's phonation, in an embodiment a voice recorder, such as a microphone.

In an embodiment, the task to be performed by the subject is as follows: Cheer the Monster requires the participant to control the speed at which the monster runs towards his goal. The monster is trying to run as far as possible in 30 seconds. Subjects are asked to make as loud an “aaah” sound as they can, for as long as possible. The volume of the sound is determined and used to modulate the character's running speed. The game duration is 30 seconds so multiple “aaah” sounds may be used to complete the game if necessary.

(3) Tap-The-Monster test:

The term “Tap the Monster test”, as used herein, relates to a test designed for the assessment of distal motor function in accordance with MFM D3 (Berard C et al. (2005), Neuromuscular Disorders 15:463). In an embodiment, the tests are specifically anchored to MFM tests 17 (pick up ten coins), 18 (go around the edge of a CD with a finger), 19 (pick up a pencil and draw loops) and 22 (place finger on the drawings), which evaluate dexterity, distal weakness/strength, and power. The game measures the participant's dexterity and movement speed. In an embodiment, the task to be performed by the subject is as follows: Subject taps on monsters appearing randomly at 7 different screen positions.

FIG. 3C show a correlations plot for analysis models, in particular regression models, for predicting a total motor score (TMS) value indicative of Huntington's disease. The input data was data from HD OLE study, ISIS 44319-CS2 from 46 subjects. The ISIS 443139-CS2 study is an Open Label Extension (OLE) for patients who participated in Study ISIS 443139-CS1. Study ISIS 443139-CS1 was a multiple-ascending dose (MAD) study in 46 patients with early manifest HD aged 25-65 years, inclusive. In total, 43 features were evaluated from one test, the Draw-A-Shape test (see above), were evaluated during model building using the method according to the present invention. The following table gives an overview of selected features used for prediction, test from which the feature was derived, short description of feature and ranking:

| Performance parameter | test | description | rank |
|---|---|---|---|
| log10 SPIRAL_sp_cov | Draw-A- | The coefficient of | 1 |
| | Shape | variation in the | |
| | | drawing velocity | |
| | | of the Spiral shape | |
| SPIRAL_hausD | Draw-A- | The maximum | 2 |
| | Shape | hausdorff distance | |
| | | between drawn and | |
| | | reference shape - | |
| | | as a proxy for | |
| | | maximumm drawing | |
| | | error for the Spiral | |
| | | shape | |
| log10 | Draw-A- | The number of way- | 3 |
| SQUARE_acc_celerity | Shape | points hit (accuracy) | |
| | | divided by the time | |
| | | take to complete | |
| | | the Square shape | |
| sigmoid | Draw-A- | | 4 |
| SQUARE_Mag_areaError | Shape | | |

FIG. 3C shows the Spearman correlation coefficient r s between the predicted and true target variables, for each regressor type, in particular from left to right for kNN, linear regression, PLS, RF and XT, as a function of the number of features f included in the respective analysis model. The upper row shows the performance of the respective analysis models tested on the test data set. The lower row shows the performance of the respective analysis models tested in training data. The curves in the lower row show results for “all” and “Mean” in the lower row are results obtained from predicting the target variable on the training data. “Mean” refers to the prediction on the average value of all observations per subject. “all” refers to the prediction on all individual observations. For assessing the performance of any machine learning model, the results from the test data (top row) were considered more reliable. It was found that the best performing regression model is PLS with 4 features included in the model, having an r s value of 0.65, indicated with circle and arrow.

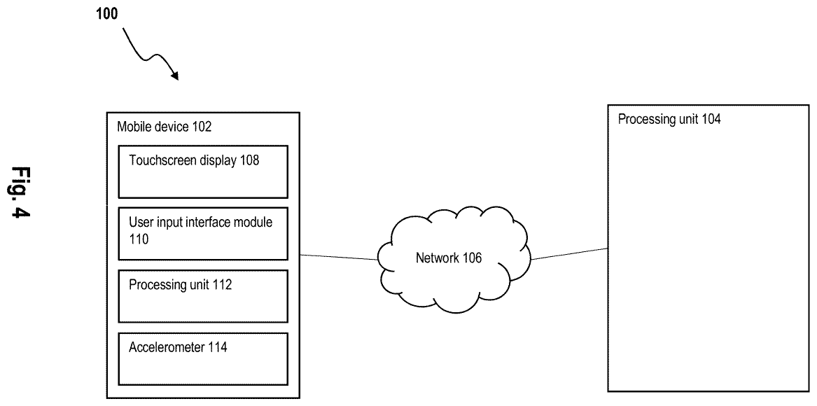

FIG. 4 onward illustrate many of the principles of the invention with regard to the pinching test features, and the overshoot/undershoot features which may be extracted from the draw-a-shape test.

FIG. 4 shows a high-level system diagram of an example arrangement of hardware which may perform the invention of the present application. System 100 includes two main components: a mobile device 102, and a processing unit 104. The mobile device 102 may be connected to processing unit 104 by network 106, which may be a wired network, or a wireless network such as a Wi-Fi or cellular network. In some implementations of the invention, the processing unit 104 is not required, and its function can be performed by processing unit 112 which is present on the mobile device 102. The mobile device 102 includes a touchscreen display 108, a user input interface module 110, a processing unit 112, and an accelerometer 114.

The system 100 may be used to implement at least one of a pinching test, and/or a draw-a-shape test, as have been described previously in this application. The aim of a pinching test is to assess fine distal motor manipulation (gripping and grasping), and control by evaluating accuracy of pinch closed finger movement. The test may cover the following aspects of impaired hand motor function: impaired gripping/grasping function, muscle weakness, and impaired hand-eye coordination. In order to perform the test, a patient is instructed to hold a mobile device in the untested hand (or by placing it on a table or other surface) and by touching the screen with two finger from the same hand (preferably the thumb+index finger/middle finger) to squeeze/pinch as many round shapes as they can during fixed time, e.g. 30 seconds. Round shapes are displayed at a random location within the game area. Impaired fine motor performance will affect the performance. The test may be performed alternatingly with the left hand and the right hand. The following terminology will be used when describing the pinching test:
Touch Events: The touch interactions recorded by the OS recording when fingers touched the screen and where the screen was touched
Start distance: the distance between the two points identified by the first taps of two fingers
Bounding box: the box containing the shape to be squeezed
Initial fingers distance: The initial distance when two fingers touches the screen
Game Area: The game area fully contains the shape to be squeezed and is delimited by a rectangle.
Game Area Padding: The padding between the screen edges and the actual game area. The shapes are not displayed in this padding area.

Any or all of the following parameters may be defined:
Bounding box height
Bounding box width
The minimum initial distance between two pointers prior to pinching
The minimum distance between two pointers to squeeze the shape.
A minimum change in separation between the fingers.

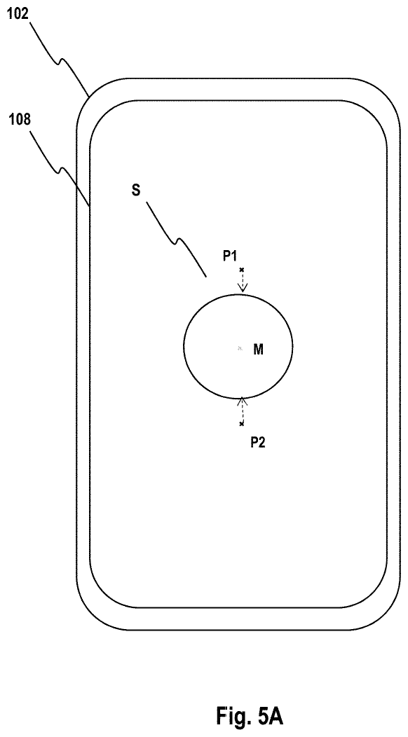

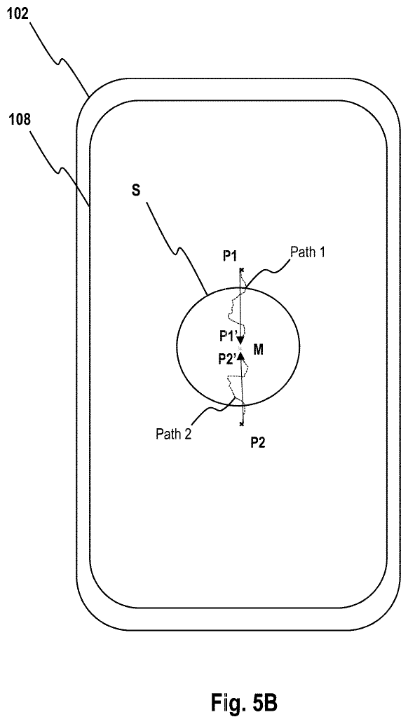

FIGS. 5A and 5B show examples of displays which a user may see when performing a pinching test. Specifically, FIG. 5A shows mobile device 102, having touchscreen display 108. The touchscreen display 108 shows a typical pinching test, in which a shape S includes two points P1 and P2. In some cases, the user will only be presented the shape S (i.e. the points P1 and P2 will not be identified specifically). A midpoint M is also shown in FIG. 5A, though this may not be displayed to the user either. In order to take the test, the user of the device must use two fingers simultaneously to “pinch” the shape S as much as possible, effectively by bringing points P1 and P2 as close as possible to each other. Preferably, a user is able to do so using two fingers only. The digital biomarker features which may be extracted from an input received by the touchscreen have been discussed earlier. Some of these are explained below with reference to FIG. 5B.

FIG. 5B shows two additional points, P1′ and P2′ which are the endpoints of Path 1 and Path 2, respectively. Path 1 and Path 2 represents the path taken by a user's fingers when performing the pinching test. Some features which may be derived from the arrangement of FIG. 5B include:
The distance between P1′ and P2′.
The average distance between P1′ and M, and the distance between P2′ and M.
The ratio of the length of Path 1, and the distance (in a straight line) between P1 and P1′
The ratio of the length of Path 2, and the distance (in a straight line) between P2 and P2′
Statistical parameters derived from the above, based on a plurality of tests.

It should be stressed that all of the features discussed earlier in the application may be used in conjunction with the system 100 shown in FIG. 5A—it is not restricted to the examples shown in FIG. 5B.

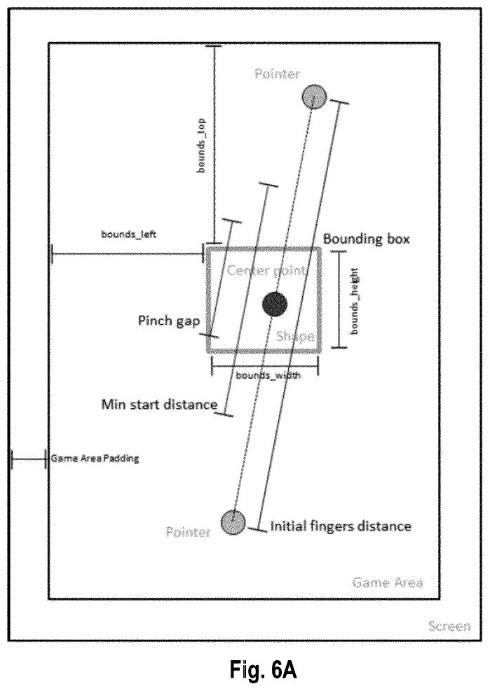

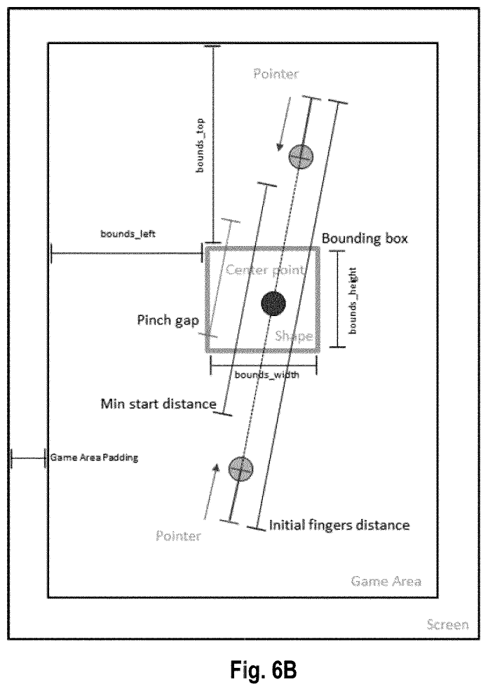

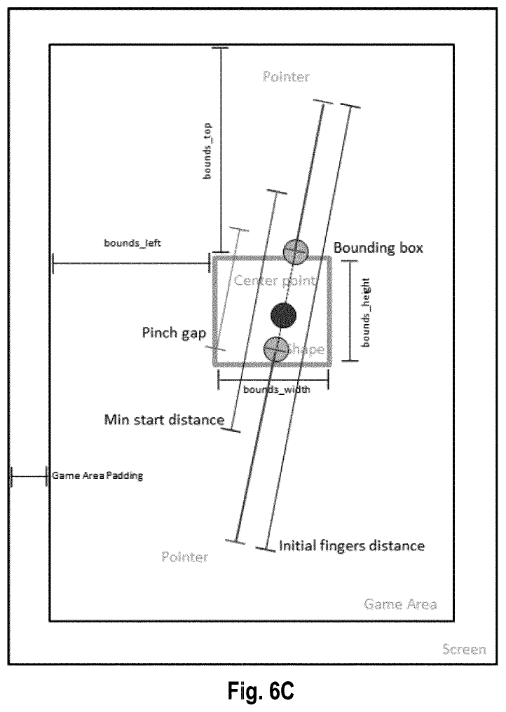

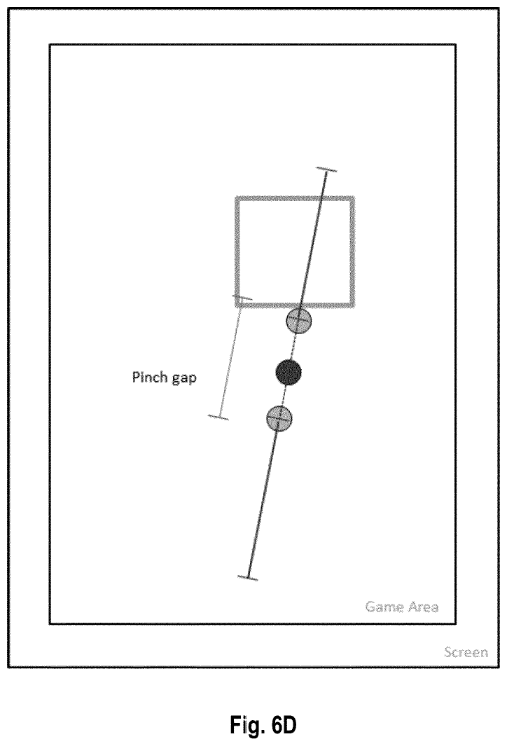

FIGS. 6A to 6D illustrate the various parameters referred to above, and examples of how these parameters may be used to determine whether the test has started, whether the test has been completed, and whether the test has been completed successfully. It should be emphasized that these conditions apply more generally than to the specific examples of the pinching test shown in the drawings. Referring now to FIG. 6A, the test may be considered to begin when: two fingers are touching the screen (as illustrated by the outermost circles in FIG. 6A), when the “Initial fingers distance” is greater than the “Minimum start distance”, when the centre point between the two fingers (the dot at the midpoint of the “Initial fingers distance”) is located within the bounding box, and/or the fingers are not moving in different directions.

We now discuss various features which can be used to determine whether a test is “complete”. For example, a test may be considered complete when the distance between the fingers is decreasing, the distance between the fingers becomes less than the pinch gap, and the distance between the fingers has decreased by at least the minimum change in separation between the fingers. In addition to determining whether the test is “complete”, the application may be configured to determine when the test is “successful”. For example, an attempt may be considered successful when the centre point between the two fingers is closer than a predetermined threshold, to the centre of the shape, or the centre of the bounding box. This predetermined threshold may be half of the pinch gap.

FIGS. 6B to 6D illustrate cases where the test is complete, incomplete, successful and unsuccessful:
In FIG. 6B, the attempt is not complete. The distance between the fingers is decreasing, and the distance between the fingers has decreased by more than the required threshold. However, the gap between the fingers is larger than the pinch gap, which means the test is not complete.
In FIG. 6C, the attempt is complete. The distance between the fingers is decreasing, the distance between the fingers is less than the pinch gap, and the separation between the fingers has decreased by more than the threshold value. In this case, the attempt is also successful, because the centre point between the fingers is less than half the pinch gap from the centre of the shape.
In FIG. 6D, the test is complete because the distance between the fingers is decreasing, the distance between the fingers is less than the pinch gap, and the separation between the fingers has decreased by greater than the threshold separation. However, the attempt is not successful, because the centre point between the fingers is further than half the pinch gap from the centre of the shape (i.e. the centre of the bounding box).

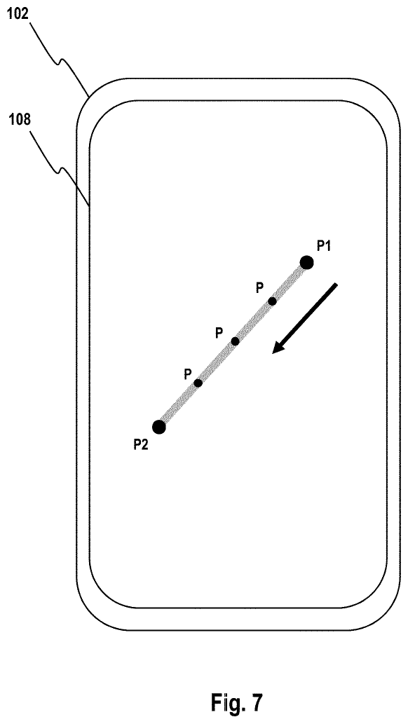

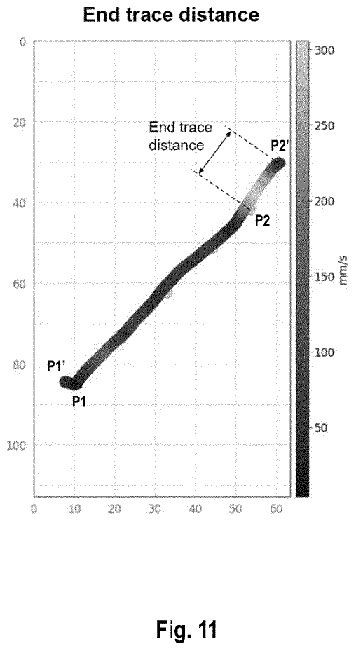

FIGS. 7 to 10 show examples of displays which a user may see when performing a draw-a-shape test. FIG. 11 onwards show results which may be derived from a user's draw-a-shape attempts and which form the digital biomarker feature data which may be inputted into the analysis model.

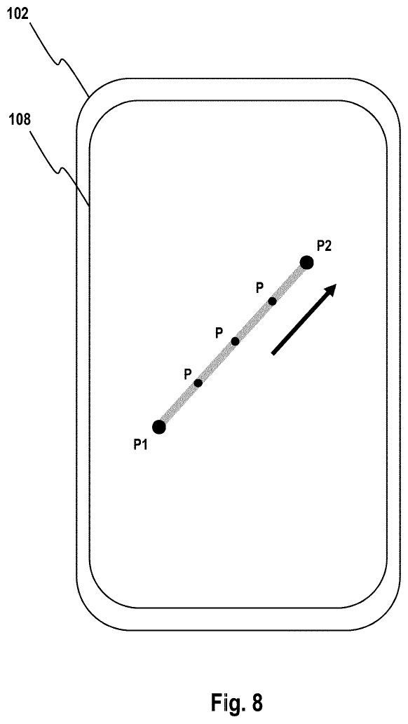

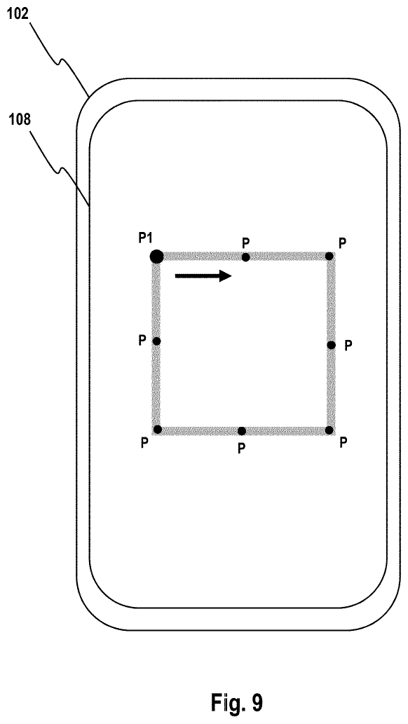

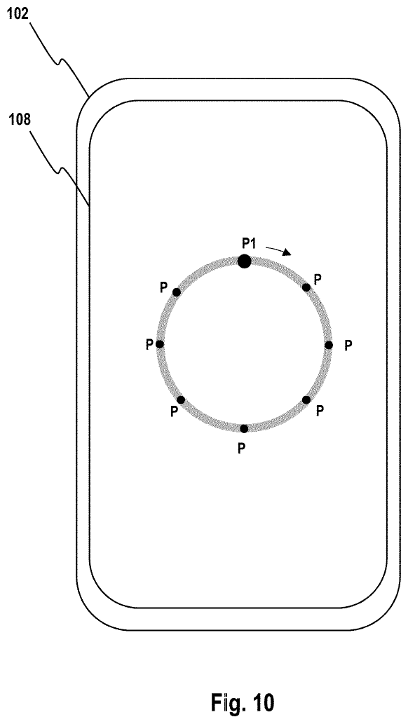

FIG. 7 shows a simple example of a draw-a-shape test in which a user has to trace a line on the touchscreen display 108 from top to bottom. The specific case of FIG. 7, the user is shown a starting point P1, an end point P2, a series of intermediate points P, and a general indication (in grey in FIG. 7) of the path to trace. In addition, the user is provided with an arrow indicating in which direction to follow the path. FIG. 8 is similar, except the user is to trace the line from bottom to top. FIGS. 9 and 10 are also similar, except in these cases, the shapes are a square and a circle respectively, which are closed. In these cases, the first point P1 is the same as the end point P1, and the arrow indicates whether the shape should be traced clockwise or anticlockwise. The present invention is not limited to lines, squares, and circles. Other shapes which may be used (as shown shortly) are figures-of-eight, and spirals.

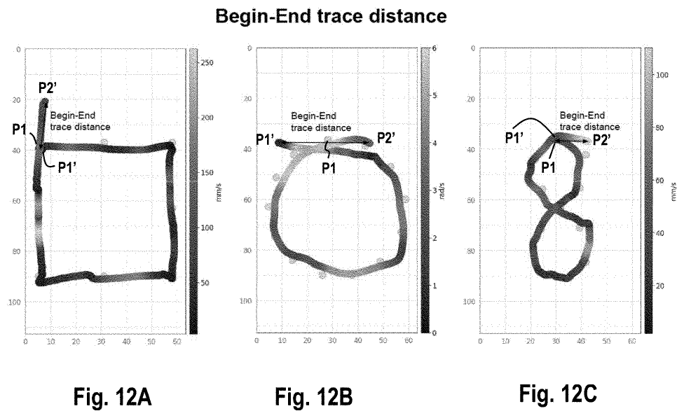

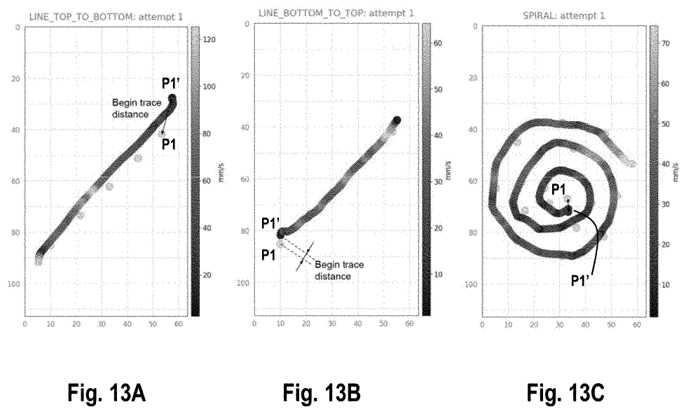

As has been discussed earlier in this application, three useful features may be extracted from draw-a-shape tests. These are illustrated in FIG. 11 onwards. FIG. 11 illustrates the feature referred to herein as the “end trace distance”, which is the deviation between the desired endpoint P2, and the endpoint P2′ of the user's path. This effectively parameterizes the user's overshoot. This a useful feature, because it provides a way of measuring a user's ability to control the endpoint of a movement, which is an effective indicator of a degree of motor control of a user. FIGS. 12A to 12C each show a similar feature, which is the “begin-end trace distance”, namely the distance between the start point of the user's path P1′ and the end point of the user's path P2′. This is a useful feature to extract from the closed shapes, such as the square, circle, and figure-of-eight shown in FIGS. 12A, 12B, and 12C, respectively, because if the test is executed perfectly, then the path should begin at the same point as it ended. The begin-end trace distance feature therefore provides the same useful information as the end trace distance, discussed previously. In addition, however, this feature also provides information about how accurately the user is able to place their finger on the desired start position P1, which tests a separate aspect of motor control too. FIGS. 13A to 13C illustrate a “begin trace distance”, which is the distance between the user's start point P1′ and the desired start point P1. As discussed, this provides information about how accurately a user is able to position their finger at the outset.

1. A computer-implemented method for quantitatively determining a clinical parameter indicative of a status or progression of a disease, the computer-implemented method comprising:
providing a distal motor test to a user of a mobile device, the mobile device having a touchscreen display, wherein providing the distal motor test to the user of the mobile device comprises:
causing the touchscreen display of the mobile device to display an image comprising: a reference start point, a reference end point, and indication of a reference path to be traced between the start point and the end point;
receiving an input from the touchscreen display of the mobile device, the input indicative of a test path traced by a user attempting to trace the reference path on the display of the mobile device, the test path comprising: a test start point, a test end point, and a test path traced between the test start point and the test end point; and
extracting digital biomarker feature data from the received input, the digital biomarker feature data comprising:
a deviation between the test end point and the reference end point;
a deviation between the test start point and the reference start point; and/or
a deviation between the test start point and the reference end point; and
wherein:
the extracted digital biomarker feature data is the clinical parameter; or
the method further comprises calculating the clinical parameter from the extracted biomarker feature data.

2. The computer-implemented method of claim 1, wherein:
the reference start point is the same as the reference end point, and the reference path is a closed path.

3. The computer-implemented method of claim 2, wherein:
the closed path is a square, a circle or a figure-of-eight.

4. The computer-implemented method of claim 1, wherein:
the reference start point is different from the reference end point, and the reference path is an open path; and
the digital biomarker feature data is the deviation between the test end point and the reference end point.

5. The computer-implemented method of claim 4, wherein:
the open path is a straight line, or a spiral.

6. The computer-implemented method of any one of claims 1 to 5, wherein:
the method comprises:
receiving a plurality of inputs from the touchscreen display, each of the plurality of inputs indicative of a respective test path traced by a user attempting to trace the reference path on the display of the mobile device, the test path comprising: a test start point, a test end point, and a test path traced between the test start point and the test end point;
extracting digital biomarker feature data from each of the plurality of received inputs, thereby generating a respective plurality of pieces of digital biomarker features data, each piece of digital biomarker feature data comprising:
a deviation between the test end point and the reference end point for the respective received input;
a deviation between the test start point and the reference start point; and/or
a deviation between the test start point and the test end point for the respective input.

7. The computer-implemented method of claim 6, wherein:
the method comprises:
deriving a statistical parameter from the plurality of pieces of digital biomarker feature data.

8. The computer-implemented method of claim 7, wherein:
the statistical parameter comprises one or more of:
a mean;
a standard deviation;
a percentile;
a kurtosis; and
a median.

9. The computer-implemented method of any one of claims 1 to 8, wherein:
the plurality of received inputs includes:
a first subset of received inputs, each indicative of a respective test path traced by a user attempting to trace the reference path on the touchscreen display of the mobile device using their dominant hand, the first subset of received inputs having a respective first subset of extracted pieces of digital biomarker data; and
a second subset of receive inputs, each indicative of a respective test path traced by a user attempting to trace the reference path on the touchscreen display of the mobile device using their non-dominant hand, the second subset of received inputs having a respective second subset of extracted pieces of digital biomarker data;
the method further comprises:
deriving a first statistical parameter corresponding to the first subset of extracted pieces of digital biomarker feature data;
deriving a second statistical parameter corresponding to the second subset of extracted pieces of digital biomarker feature data; and
calculating a handedness parameter by calculating the difference between the first statistical parameter and the second statistical parameter, and optionally dividing the difference by the first statistical parameter or the second statistical parameter.

10. The computer-implemented method of any one of claims 1 to 9, wherein:
the plurality of received inputs includes:
a first subset of received inputs, each indicative of a respective test path traced by a user attempting to trace the reference path on the touchscreen display of the mobile device in a first direction, the first subset of received inputs having a respective first subset of extracted pieces of digital biomarker data; and
a second subset of receive inputs, each indicative of a respective test path traced by a user attempting to trace the reference path on the touchscreen display of the mobile device in a second direction, opposite form the first direction, the second subset of received inputs having a respective second subset of extracted pieces of digital biomarker data;
the method further comprises:
deriving a first statistical parameter corresponding to the first subset of extracted pieces of digital biomarker feature data;
deriving a second statistical parameter corresponding to the second subset of extracted pieces of digital biomarker feature data; and
calculating a directionality parameter by calculating the difference between the first statistical parameter and the second statistical parameter, and optionally dividing the difference by the first statistical parameter or the second statistical parameter.

11. The computer-implemented method of any one of claims 1 to 10, wherein:
the disease whose status is to be predicted is multiple sclerosis and the clinical parameter comprises an expanded disability status scale (EDSS) value,
the disease whose status is to be predicted is spinal muscular atrophy and the clinical parameter comprises a forced vital capacity (FVC) value, or
wherein the disease whose status is to be predicted is Huntington's disease and the clinical parameter comprises a total motor score (TMS) value.

12. The computer-implemented method of any one of claims 1 to 11, further comprising:
applying at least one analysis model to the digital biomarker feature data or a statistical parameter derived from the digital biomarker feature data; and
predicting a value of the at least one clinical parameter based on the output of the at least one analysis model.

13. The computer-implemented method of claim 13, wherein:
the analysis model comprises a trained machine learning model.

14. The computer-implemented method of claim 14, wherein:
the analysis model is a regression model, and the trained machine learning model comprises one or more of the following algorithms:
a deep learning algorithm;
k nearest neighbours (kNN);
linear regression;
partial last-squares (PLS);
random forest (RF); and
extremely randomized trees (XT).

15. The computer implemented method of claim 14, wherein:
the analysis model is a classification model, and the trained machine learning model comprises one or more of the following algorithms:
a deep learning algorithm;
k nearest neighbours (kNN);
support vector machines (SVM);
linear discriminant analysis;
quadratic discriminant analysis (QDA);
naïve Bayes (NB);
random forest (RF); and
extremely randomized trees (XT).

16. A computer-implemented method of determining a status or progression of a disease, the computer-implemented method comprising the steps of:
executing the computer-implemented method of any one of claims 1 to 15; and
determining the status or progression of the disease based on the determined clinical parameter.

17. A system for quantitatively determining a clinical parameter indicative of a status or progression of a disease, the system including:
a mobile device having a touchscreen display, a user input interface, and a first processing unit; and
a second processing unit;
wherein:
the mobile device is configured to provide a distal motor test to a user thereof, wherein providing the distal motor test comprises:
the first processing unit causing the touchscreen display of the mobile device to display an image comprising: a reference start point, a reference end point, and indication of a reference path to be traced between the start point and the end point;
the user input interface is configured to receive from the touchscreen display, an input indicative of a test path traced by a user attempting to trace the reference path on the display of the mobile device, the test path comprising: a test start point, a test end point, and a test path traced between the test start point and the test end point; and
the first processing unit or the second processing unit is configured to extract digital biomarker feature data from the received input, the digital biomarker feature data comprising:
a deviation between the test end point and the reference end point; and/or
a deviation between the test start point and the test end point; and
wherein:
the extracted digital biomarker feature data is the clinical parameter; or
the first processing unit or the second processing unit is further configured to calculate the clinical parameter from the extract digital biomarker feature data.

18. A system for determining a status or progression of a disease, the system comprising;
a mobile device having a touchscreen display, a user input interface, and a first processing unit; and
a second processing unit;
wherein:
the mobile device is configured to provide a distal motor test to a user thereof, wherein providing the distal motor test comprises:
the first processing unit causing the touchscreen display of the mobile device to display an image comprising: a reference start point, a reference end point, and indication of a reference path to be traced between the start point and the end point;
the user input interface is configured to receive from the touchscreen display, an input indicative of a test path traced by a user attempting to trace the reference path on the display of the mobile device, the test path comprising: a test start point, a test end point, and a test path traced between the test start point and the test end point; and
the first processing unit or the second processing unit is configured to extract digital biomarker feature data from the received input, the digital biomarker feature data comprising:
a deviation between the test end point and the reference end point; and/or
a deviation between the test start point and the test end point; and
wherein:
the extracted digital biomarker feature data is the clinical parameter; or
the first processing unit or the second processing unit is further configured to calculate the clinical parameter from the extract digital biomarker feature data; and
the first processing unit or the second processing unit is configured to determine the status or progression of the disease based on the determined clinical parameter.
